(19) United States
(12) Patent Application Publication
(10) Pub. No.: US 2025/0120978 A1
(43) Pub. Date: Apr. 17, 2025

(54) JAK INHIBITOR WITH ERK1/2 AND/OR SHP2 INHIBITORS COMBINATION THERAPY

(71) Applicant: Erasca, Inc., San Diego, CA (US)

(72) Inventors: Leenus MARTIN, San Diego, CA (US); Robert Field SHOEMAKER, San Diego, CA (US); Leslie Harris BRAIL, San Diego, CA (US); Erin Denise LEW, San Diego, CA (US)

(21) Appl. No.: 18/692,508

(22) PCT Filed: Sep. 16, 2022
(86) PCT No.: PCT/US2022/043891

§ 371 Date: Mar. 15, 2024

Related U.S. Application Data
(60) Provisional application No. 63/245,642, filed on Sep. 17, 2021.

(51) Int. Cl. A61K 31/5383; A61K 31/506; A61K 31/519; A61P 35/02

(52) U.S. Cl. CPC A61K 31/5383; A61K 31/506; A61K 31/519; A61P 35/02

(57) ABSTRACT

The present invention relates generally to the use an ERK1/2 inhibitor or a SHP2 inhibitor alone or in combination with a JAK inhibitor for treating cancer, specifically liquid tumors such as a myeloproliferative neoplasm (MPN).

### CROSS-REFERENCE

This application claims the benefit of U.S. Provisional Application Ser. No. 63/245,642 filed Sep. 17, 2021 which is hereby incorporated by reference in its entirety.

### BACKGROUND OF THE INVENTION

ERK1 and ERK2 (collectively “ERK1/2”) are related protein-serine/threonine kinases that participate in, amongst others, the Ras-Raf-MEK-ERK signal transduction pathway, which is sometimes denoted as the mitogen-activated protein kinase (MAPK) pathway. This pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

Chronic Myeloproliferative Neoplasms (MPNs) are clonal hematopoietic disorders of hematopoietic multipotent stem/progenitor cells. They include polycythemia vera (PV), essential thrombocythemia (ET) and primary myelofibrosis (PMF). The JAK2V617F mutation is commonly found in (MPNs) and results in constitutive activation of JAK-STAT signaling pathway. The JAK2V617F mutation is found in approximately 65-70% of these three disorders and is present in 95% of PVs. The JAK2 inhibitor ruxolitinib is the first approved targeted therapy in this field. Although ruxolitinib is known for reduction in symptom burden, there are many clinical challenges that still need to be overcome. Therefore, novel combination therapies may provide a clinical benefit.

### SUMMARY OF THE INVENTION

ERK1/2 appear to be activated by MEK through phosphorylation of both a threonine and a tyrosine residue, namely at Tyr204/187 and Thr202/185. Once activated, ERK1/2 catalyze the phosphorylation of serine/threonine residues of more than 100 substrates and activate both cytosolic and nuclear proteins that are linked to cell growth, proliferation, survival, angiogenesis and differentiation, all hallmarks of the cancer phenotype. Thus it may be beneficial to target ERK 1 and ERK 2 to develop and use ERK1/2 inhibitors as a way to inhibit tumor growth.

Furthermore, an ERK inhibitor may have utility in combination with other kinase, for example MAPK, inhibitors. Recently, researchers reported that dual inhibition of MEK and ERK by small molecule inhibitors was synergistic and acted to overcome acquired resistance to MEK inhibitors. See Hatzivassiliou et al., ERK Inhibition Overcomes Acquired Resistance to MEK Inhibition, Mol. Cancer Ther. 2012, 11, 1143-1154.

In addition to ERK1/2, SHP2 also operates upstream of the RAS pathway. SHP2 is a protein tyrosine phosphatase and a key positive regulator of the growth signals from the RTK growth factor receptors to the intracellular signaling pathways (including RAS/MAPK and PI3K) that promote growth and survival of normal cells and cancer cells. As such, SHP2 is a convergent node for upstream RTK signaling: activated SHP2 upregulates (“turns up”) the positive signals and downregulates (“turns down”) the negative signals in the signaling cascades. SHP2 also serves as a central node in relaying the growth and survival signals from RTKs such as EGFR and FLT3 to RAS/MAPK and other intracellular pathways. SHP2 is an attractive target because SHP2 inhibition ubiquitously blocks the growth signals from multiple RTKs, preventing cancer cells from bypassing the blockade on a specific RTK (e.g., EGFR inhibitor) through activation of other RTK growth factor receptors (e.g., MET).

The Janus kinase (JAK) family is one of ten recognized families of non-receptor tyrosine kinases. Mammals have four members of this family, JAK 1, JAK 2, JAK 3, and Tyrosine kinase 2 (Tyk2). Each protein has a kinase domain and a catalytically inactive pseudo-kinase domain, and they each bind cytokine receptors through amino-terminal FERM (Band-4.1, ezrin, radixin, moesin) domains. Upon binding of cytokines to their receptors, JAK s are activated and phosphorylate the receptors, creating docking sites for signaling molecules, especially members of the signal transducer and activator of transcription (Stat) family. Mutations of the Drosophila JAK (Hopscotch) have revealed developmental defects, and constitutive activation of JAK s in flies and humans is associated with leukemia-like syndromes. Through the generation of JAK-deficient cell lines and gene-targeted mice, the essential, nonredundant functions of JAK s in cytokine signaling have been established.

The opportunity to target signal transduction pathways from multiple angles and potentially ameliorate feedback loops upstream of Ras via ERK1/2 and SHP2 and additionally in combination via JAK provides opportunities for developing methods that employ combination therapies.

The present embodiments disclosed herein generally relate to compositions and methods related to combination therapies to treat cancer utilizing an ERK1/2 inhibitor in conjunction with a JAK, or a SHP2 inhibitor in conjunction with a JAK inhibitor while providing an unexpected degree of synergy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 is a bar graph showing cell viability of Human JAK2V617F-positive UKE-1 cells versus concentration of Compound 2, ruxolitinib, alone and in combination.

FIG. 2 is a bar graph showing cell viability of Human JAK2V617F-positive UKE-1 cells versus concentration of Compound 1, ruxolitinib, alone and in combination.

FIG. 3 is a bar graph showing cell viability of Human JAK2V617F-positive HEL cells versus concentration of Compound 2, ruxolitinib, alone and in combination.

FIG. 4 is a bar graph showing cell viability of Human JAK2V617F-positive HEL cells versus concentration of Compound 1, ruxolitinib, alone and in combination.

FIG. 5 is a bar graph showing percent apoptosis of Human JAK2V617F-positive UKE-1 cells versus concentration of Compound 2, ruxolitinib, alone and in combination.

FIG. 6A is a photograph showing spleen size in in Jak2V617F mice in the presence of vehicle, Compound 2, ruxolitinib, and the combination of Compound 2 with ruxolitinib.

FIG. 6B is a bar graph showing spleen weight (mg) in in Jak2V617F mice in the presence of vehicle, Compound 2, ruxolitinib, and the combination of Compound 2 with ruxolitinib.

FIG. 7A is a bar graph showing hematopoietic progenitor colonies in MPN patient CD34+ cells as percentage of control versus concentration of Compound 2, ruxolitinib, alone and in combination in a first patient sample.

FIG. 7B is a bar graph showing hematopoietic progenitor colonies in MPN patient CD34+ cells as percentage of control versus concentration of Compound 2, ruxolitinib, alone and in combination in a second patient sample.

FIG. 7C is a bar graph showing hematopoietic progenitor colonies in MPN patient CD34+ cells as percentage of control versus concentration of Compound 2, ruxolitinib, alone and in combination in a third patient sample.

FIG. 7D is a bar graph showing hematopoietic progenitor colonies in MPN patient CD34+ cells as percentage of control versus concentration of Compound 2, ruxolitinib, alone and in combination in a fourth patient sample.

FIG. 8 is a bar graph showing hematopoietic progenitor colonies in MPN patient CD34+ cells as percentage of control versus concentration of Compound 1, ruxolitinib, alone and in combination in a patient sample.

### DETAILED DESCRIPTION OF THE INVENTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent, or improve an unwanted condition or disease of a patient. In some embodiments, a therapeutic agent such as a compound 1 is directed to the treatment and/or the amelioration of cancers.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable,” it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

The term “substantially the same as” as used herein, refers to a powder x-ray diffraction pattern or differential scanning calorimetry pattern that is non-identical to those depicted herein, but that falls within the limits of experimental error, when considered by one of ordinary skill in the art.

### ERK1/2 Inhibitor

In some embodiments, the ERK1/2 inhibitor is LTT462, ulixertinib (BVD-523), MK-83 ravoxertinib, ASTX029, SCH772984, KO-947, AZD0364 (ATG-017), CC-90003, ortemuterkib (LY3214996).

In some embodiments, the ERK1/2 inhibitor is compound 1, or a pharmaceutically acceptable salt thereof.

### Compound 1

Disclosed herein is (S)—N-(2-amino-1-(3-chloro-5-fluorophenyl)ethyl)-1-(5-methyl-2-((tetrahydro-2H-pyran-4-yl)amino)pyrimidin-4-yl)-1H-imidazole-4-carboxamide:

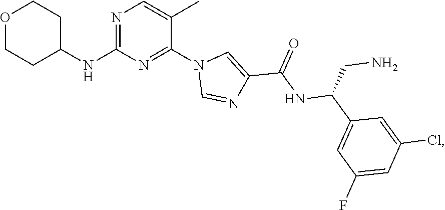 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

In some embodiments, the salt of compound 1 is the mandelic acid salt. In some embodiments, the salt of compound 1 is the benzenesulfonic acid salt. In some embodiments, the salt of compound 1 is the hydrochloride salt. In some embodiments, the salt of compound 1 is the p-toluenesulfonic acid salt.

In some embodiments, the salt of compound 1 is the benzenesulfonic acid salt.

### SHP2 Inhibitor

SHP2 plays important roles in fundamental cellular functions including proliferation, differentiation, cell cycle maintenance and motility, and regulates multiple intracellular signaling pathways in response to wide range of growth factors, cytokines, and hormones. Cell signaling processes in which SHP2 participates include MAPK, PI3K and JAK pathways. SHP2 inhibitors have the potential to attenuate upstream RTK signaling that often drives oncogenic signaling and adaptive tumor escape globally, and to become a broad-spectrum anticancer drug.

In some embodiments, the SHP2 inhibitor is sodium stibogluconate, RMC-4550, NSC87877, SPI-112, TNO155, IACS-13909, or SHP099 HCl.

In some embodiments, the SHP2 inhibitor is compound 2, or a pharmaceutically acceptable salt thereof.

### Compound 2

Disclosed herein is (3-((3S,4S)-4-amino-3-methyl-2-oxa-8-azaspiro[4.5]decan-8-yl)-6-(((6aS,8S)-8-((methoxymethoxy)methyl)-6a,7,8,9-tetrahydro-6H-pyrido[3,2-b]pyrrolo[1,2-d][1,4]oxazin-4-yl)thio)pyrazin-2-yl)methanol:

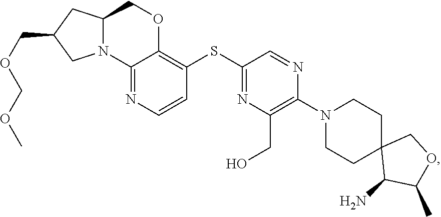 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

### JAK Inhibitor

Altered production of cytokines can result in pathologies ranging from autoimmune diseases to malignancies. The Janus Kinases family is a small group of receptor-associated signaling molecules that is essential to the signal cascade originating from type I and type II cytokine receptors. Inhibition of tyrosine kinases enzymatic activity using small molecules has recently become a powerful tool for treatment of several malignancies.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib. Ruxolitinib is sold in the US under the brand name JAKAFI®, and elsewhere in the world, under the brand name JAKAVI®.

### Combinations

Disclosed herein is a method of treating cancer in a subject in need thereof, the method comprising administering to the subject in need thereof a therapeutically effective amount of
(i) compound 1:

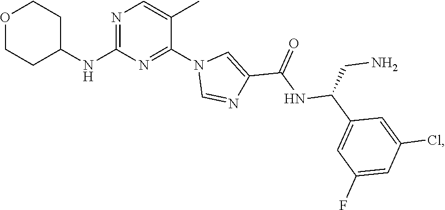 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

In some embodiments, the method further comprises administering a SHP2 inhibitor. In some embodiments, the SHP2 inhibitor is sodium stibogluconate, RMC-4550, NSC87877, SPI-112, TNO155, IACS-13909, or SHP099 HCl.

In some embodiments, the SHP2 inhibitor is compound 2:

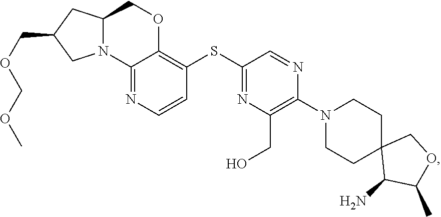 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Disclosed herein is a method of treating cancer in a subject in need thereof, the method comprising administering to the subject in need thereof a therapeutically effective amount of
(i) compound 2:

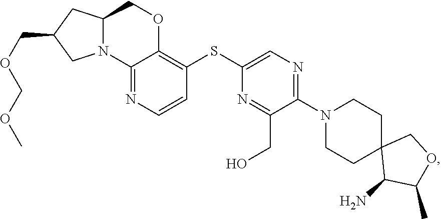 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(ii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

In some embodiments, the method further comprises administering an ERK1/2 inhibitor. In some embodiments, the ERK1/2 inhibitor is LTT462, ulixertinib (BVD-523), MK-8353, ravoxertinib, ASTX029, SCH772984, KO-947, AZD0364 (ATG-017), CC-90003, or temuterkib (LY3214996).

In some embodiments, the ERK1/2 inhibitor is compound 1:

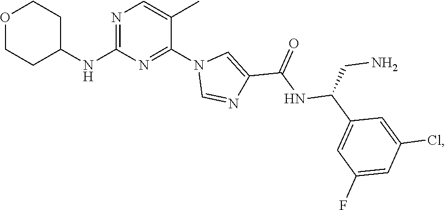 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

Disclosed herein is a method of treating cancer in a subject in need thereof, the method comprising administering to the subject in need thereof a therapeutically effective amount of
(i) compound 2:

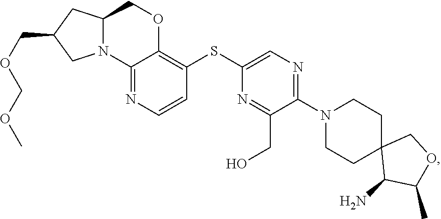 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(ii) an ERK1/2 inhibitor; and
(iii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

In some embodiments, the ERK1/2 inhibitor is LTT462, ulixertinib (BVD-523), Mk-8353, ravoxertinib, ASTX029, SCH772984, KO-947, AZD0364 (ATG-017), CC-90003, ortemuterkib (LY3214996).

In some embodiments, the ERK1/2 inhibitor is compound 1:

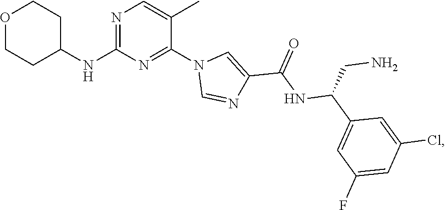 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

Disclosed herein is a method of treating cancer in a subject in need thereof, the method comprising administering to the subject in need thereof a therapeutically effective amount of
(i) compound 1:

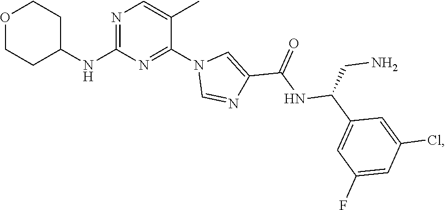 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) a SHP2 inhibitor; and
(iii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

In some embodiments, the SHP2 inhibitor is sodium stibogluconate, RMC-4550, NSC87877, SPI-112, TNO155, IACS-13909, or SHP099 HCl.

In some embodiments, the SHP2 inhibitor is compound 2:

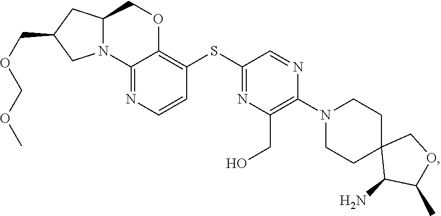 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Disclosed herein is a method of treating cancer in a subject in need thereof, the method comprising administering to the subject in need thereof a therapeutically effective amount of
(i) compound 1:

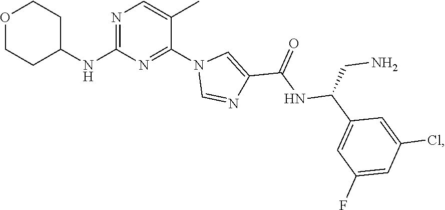 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

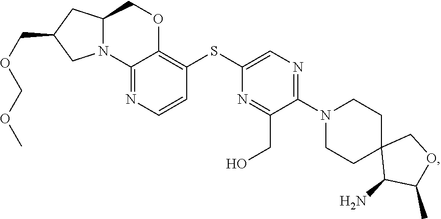 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

Disclosed herein is a method of treating cancer in a subject in need thereof, the method comprising administering to the subject in need thereof a therapeutically effective amount of
(i) compound 1:

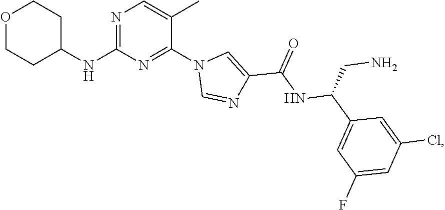 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) a SHP2 inhibitor; and
(iii) ruxolitinib.

In some embodiments, the SHP2 inhibitor is sodium stibogluconate, RMC-4550, NSC87877, SPI-112, TNO155, IACS-13909, or SHP099 HCl.

In some embodiments, the SHP2 inhibitor is compound 2:

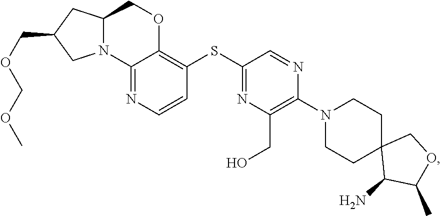 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Disclosed herein is a method of treating cancer in a subject in need thereof, the method comprising administering to the subject in need thereof a therapeutically effective amount of
(i) compound 1:

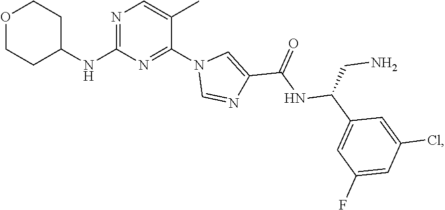 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) ruxolitinib.

Disclosed herein is a method of treating cancer in a subject in need thereof, the method comprising administering to the subject in need thereof a therapeutically effective amount of
(i) compound 2:

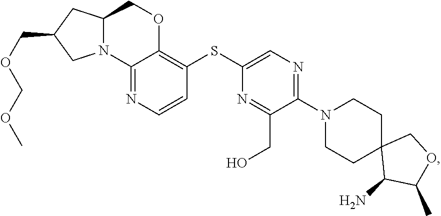 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof;
(ii) an ERK1/2 inhibitor; and
(iii) ruxolitinib.

In some embodiments, the ERK1/2 inhibitor is LTF462, ulixertinib (BVD-523), MK-8353, ravoxertinib, ASTX029, SCH772984, KO-947, AZD0364 (ATG-017), CC-90003, ortemuterkib (LY3214996).

In some embodiments, the ERK1/2 inhibitor is compound 1:

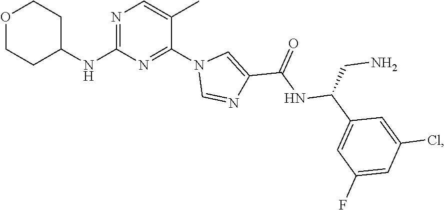 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

Disclosed herein is a method of treating cancer in a subject in need thereof, the method comprising administering to the subject in need thereof a therapeutically effective amount of
(i) compound 2:

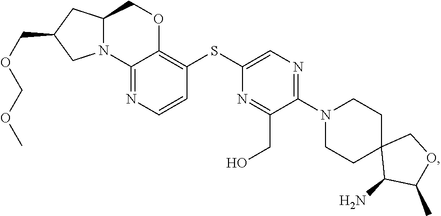 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(ii) ruxolitinib.

Disclosed herein is a method of treating cancer in a subject in need thereof, the method comprising administering to the subject in need thereof a therapeutically effective amount of
(i) compound 1:

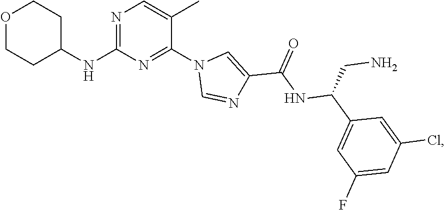 [SMILES: Cc1cnc(NC2CCOCC2)nc1-n1cnc(C(=O)N[C@H](CN)c2cc(F)cc(Cl)c2)c1]

or a pharmaceutically acceptable salt thereof;
(ii) compound 2:

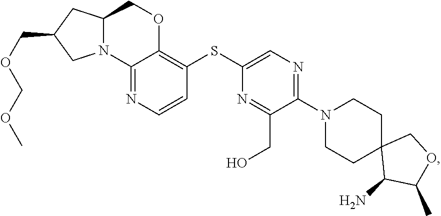 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(iii) ruxolitinib.

Disclosed herein is a method of treating a hematologic cancer in a subject in need thereof, the method comprising administering to the subject in need thereof a therapeutically effective amount of amount of compound 1:

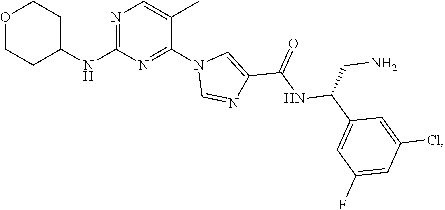 [SMILES: Cc1cnc(NC2CCOCC2)nc1-n1cnc(C(=O)N[C@H](CN)c2cc(F)cc(Cl)c2)c1]

or a pharmaceutically acceptable salt thereof.

Disclosed herein is a method of treating a hematologic cancer in a subject in need thereof, the method comprising administering to the subject in need thereof a therapeutically effective amount of amount of compound 2:

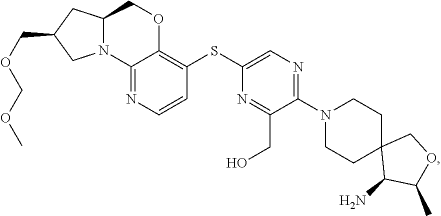 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof.

Disclosed herein is a method of treating a hematologic cancer in a subject in need thereof, the method comprising administering to the subject in need thereof a therapeutically effective amount of
(i) compound 2:

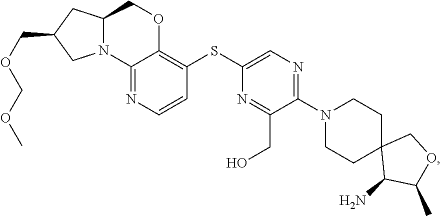 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(ii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

Disclosed herein is a method of treating a hematologic cancer in a subject in need thereof, the method comprising administering to the subject in need thereof a therapeutically effective amount of
(i) compound 1:

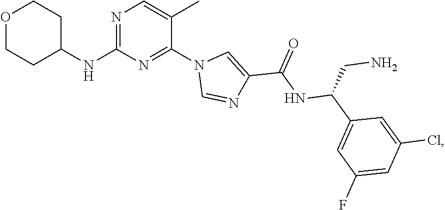 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) a JAK inhibitor.

In some embodiments, the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

In some embodiments, the JAK inhibitor is ruxolitinib.

Disclosed herein is a method of treating a hematologic cancer in a subject in need thereof, the method comprising administering to the subject in need thereof a therapeutically effective amount of
(i) compound 2:

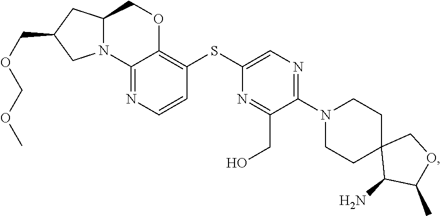 [SMILES: COCOC[C@H]1C[C@H]2COc3c(Sc4cnc(N5CCC6(CC5)CO[C@@H](C)[C@H]6N)c(CO)n4)ccnc3N2C1]

or a pharmaceutically acceptable salt thereof; and
(ii) ruxolitinib.

Disclosed herein is a method of treating a hematologic cancer in a subject in need thereof, the method comprising administering to the subject in need thereof a therapeutically effective amount of
(i) compound 1:

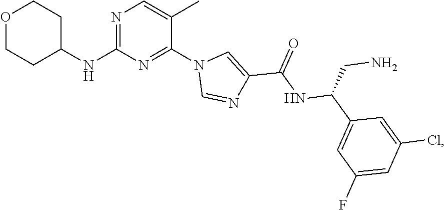 [SMILES: Cc1cnc(NC2CCOCC2)nc1-n1cnc(C(=O)N[C@H](CN)c2cc(F)cc(Cl)c2)c1]

or a pharmaceutically acceptable salt thereof; and
(ii) ruxolitinib.

### Further Combination

In some embodiments, the method disclosed herein comprises administering an additional MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor. Exemplary MAPK pathway inhibitors include, without limitation, adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, panitumumab, selumetinib, sotorasib, trametinib, ulixertinib, vandetanib, and vemurafenib.

In some embodiment the additional MAPK pathway inhibitor is adagrasib. In some embodiment the additional MAPK pathway inhibitor is afatinib. In some embodiment the additional MAPK pathway inhibitors is binimetinib. In some embodiment the additional MAPK pathway inhibitor is cetuximab. In some embodiment the additional MAPK pathway inhibitor is cobimetinib. In some embodiment the additional MAPK pathway inhibitor is dabrafenib. In some embodiment the additional MAPK pathway inhibitor is dacomitinib. In some embodiment the additional MAPK pathway inhibitor is encorafenib. In some embodiment the additional MAPK pathway inhibitor is erlotinib. In some embodiment the additional MAPK pathway inhibitor is gefitinib. In some embodiment the additional MAPK pathway inhibitor is gilteritinib. In some embodiment the additional MAPK pathway inhibitor is lapatinib. In some embodiment the additional MAPK pathway inhibitor is LTT462. In some embodiment the additional MAPK pathway inhibitor is LY3214996. In some embodiment the additional MAPK pathway inhibitor is necitumumab. In some embodiment the additional MAPK pathway inhibitor is neratinib. In some embodiment the additional MAPK pathway inhibitor is nimotuzumab. In some embodiment the additional MAPK pathway inhibitor is osimertinib. In some embodiment the additional MAPK pathway inhibitor is palbociclib. In some embodiment the additional MAPK pathway inhibitor is panitumumab. In some embodiment the additional MAPK pathway inhibitor is selumetinib. In some embodiment the additional MAPK pathway inhibitor is sotorasib. In some embodiment the additional MAPK pathway inhibitor is trametinib. In some embodiment the additional MAPK pathway inhibitor is ulixertinib. In some embodiment the additional MAPK pathway inhibitor is vandetanib.

### Cancers

Disclosed herein are methods of treating cancer using a combination disclosed herein. “Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In some embodiments the cancer is colorectal cancer (CRC), pancreatic ductal adenocarcinoma (PDAC), cholangiocarcinoma cancer, appendiceal cancer, gastric cancer, esophageal cancer, non-small cell lung cancer (NSCLC), head and neck cancer, ovarian cancer, uterine cancer, acute myeloid leukemia (AML), or melanoma. In some embodiments, the cancer is a blood cancer. In some embodiments, the blood cancer is myeloproliferative neoplasm (MPN). In some embodiments, the MPN is polycythemia vera (PV). In some embodiments, the MPN is essential thrombocythemia (ET). In some embodiments, the MPN is primary myelofibrosis (PMF).

In some embodiments, the cancer has a class 1 B-Raf mutation.

In some embodiments, the cancer harbors at least one of a EGFR, KRAS, BRAF (e.g., BRAF class III) and/or NF1 (e.g., loss of function) mutations.

In some embodiments, the cancer has a mutation in JAK2. In some embodiments, the cancer has a mutation in CALR. In some embodiments, the cancer has a mutation in MPL. In some embodiments, the cancer has any combination of mutations in JAK2, CALR, and MPL. In some embodiments, the cancer has a JAK2V617F-positive mutation. In some embodiments, the cancer has a chromosomal translocation producing a BCR/ABL fusion gene indicative of Philadelphia chromosome positive MPN chronic myeloid leukemia (CML).

In some embodiments, the mutant B-Raf comprises a V600 mutation. In some embodiments, the mutant of B-Raf comprises the mutation V600E. In some embodiments, the mutation is V600K. In some embodiments, the mutation is V600D. In some embodiments, the mutation is V600L. In some embodiments, the mutation is V600R. In some embodiments, the cancer is a BRAF V600E or V600K mutant tumor.

In some embodiments, the cancer is a mitogen-activated protein kinase (MAPK) pathway driven cancer.

In some embodiments, the cancer is a BRAF-driven cancer, HRAS-driven cancer, or a NRAS-driven cancer.

In some embodiments, the cancer comprises at least one cancer cell driven by deregulated ERK.

In some embodiments, the cancer has at least one mutation in RAS. In some embodiments, the cancer has at least one mutation in RAF. In some embodiments, the cancer has at least one mutation in MEK.

In some embodiments, the cancer has a G12C KRAS mutation. In some embodiments, the cancer has a G12D KRAS mutation. In some embodiments, the cancer has a G12S KRAS mutation. In some embodiments, the cancer has a G12V KRAS mutation. In some embodiments, the cancer has a G13D KRAS mutation. In some embodiments, the cancer has a Q16H KRAS mutation. In some embodiments, the cancer has a Q16K KRAS mutation. In some embodiments, the cancer has a Q61R NRAS mutation.

In some embodiments, the cancer is a MAPKm/MAPKi-naïve pan cancer.

In some embodiments, the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S. In some embodiments, the cancer comprises one or more EGFR mutation selected from the group consisting of L858R, exon 19 deletion, and T790M.

In some embodiments, the cancer is a liquid tumor. In some embodiments, the cancer is a solid tumor.

In some embodiments, the cancer is acute lymphoblastic leukemia (ALL), acute myeloid leukemia (AML), bladder cancer, bone cancer, breast cancer, chronic lymphocytic leukemia (CLL), chronic myeloid leukemia (CML), colorectal cancer (CRC), diffuse large b-cell lymphoma, endometrial cancer, eosinophilic myeloid neoplasm, esophageal cancer, essential thrombocythemia (ET), fallopian tube cancer, glioblastoma, glioma, head and neck cancer, hematologic neoplasm, hematopoietic neoplasm, Hodgkin's lymphoma, inflammatory breast cancer (IBC), leukemia, lung cancer, lymphoma, melanoma, multiple myeloma, myelofibrosis (MF), myelomonocytic leukemia, myeloproliferative disorder, a myeloproliferative neoplasm (MPN), NK-cell lymphoma, non-Hodgkin's lymphoma, non-small cell lung carcinoma (NSCLC), ovarian cancer, pancreatic cancer, polycythemia vera (PV), prostate cancer, salivary gland tumor, squamous cell carcinoma, T-cell leukemia, T-cell lymphoma, thyroid cancer, or urothelial carcinoma.

In some embodiments, the cancer is a hematologic cancer. In some embodiments, the hematologic cancer is acute lymphoblastic leukemia (ALL), acute myeloid leukemia (AML), chronic lymphocytic leukemia (CLL), chronic myeloid leukemia (CML), diffuse large b-cell lymphoma, hematologic neoplasm, hematopoietic neoplasm, Hodgkin's lymphoma, leukemia, lymphoma, multiple myeloma, myelofibrosis (MF), myelomonocytic leukemia, myeloproliferative disorder, a myeloproliferative neoplasm (MPN), NK-cell lymphoma, non-Hodgkin's lymphoma, polycythemia vera (PV), T-cell leukemia, or T-cell lymphoma.

In some embodiments, the hematologic cancer is leukemia, lymphoma, or multiple myeloma. In some embodiments, the hematologic cancer is leukemia. In some embodiments, the hematologic cancer is lymphoma. In some embodiments, the hematologic cancer is multiple myeloma.

In some embodiments, the hematologic cancer is a myeloproliferative neoplasm (MPN). In some embodiments, the myeloproliferative neoplasm is chronic myelogenous leukemia (CML), polycythemia vera (PV), primary myelofibrosis, essential thrombocythemia, chronic neutrophilic leukemia, or chronic eosinophilic leukemia.

In some embodiments, the hematologic cancer is myelofibrosis. In some embodiments, the hematologic cancer is intermediate or high-risk myelofibrosis. In some embodiments, the intermediate or high-risk myelofibrosis comprises primary myelofibrosis, post-polycythemia vera myelofibrosis, or post-essential thrombocythemia myelofibrosis.

### Dosing/Administration

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for the compounds disclosed herein can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising a compound disclosed herein corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

Administration of a compound disclosed herein, and combination partners described herein are at a dosage described herein or at other dose levels and compositions determined and contemplated by a medical practitioner. In certain embodiments, a compound disclosed herein, is administered for prophylactic and/or therapeutic treatments. In certain therapeutic applications, a compound disclosed herein, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In prophylactic applications, the compositions described herein are administered to a patient susceptible to or otherwise at risk of a particular disease, e.g., cancer. Such an amount is defined to be a “prophylactically effective amount or dose.” In this use, the precise amounts also depend on the patient's age, state of health, weight, and the like. When used in a patient, effective amounts for this use will depend on the risk or susceptibility of developing the particular disease, previous therapy, the patient's health status and response to the compositions, and the judgment of the treating physician.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, the compounds disclosed herein are administered in a cycle. The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

### Compound 1

In some embodiments, compound 1, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 1, or a pharmaceutically acceptable salt thereof, is administered to a subject who is in a fasted state for at least 8 hours. In other embodiments, compound 1, or a pharmaceutically acceptable salt thereof, and combination partners described herein, are administered to a subject who is in a fasted state for at least 10 hours. In yet other embodiments, compound 1, or a pharmaceutically acceptable salt thereof, and combination partners described herein, are administered to a subject who is in a fasted state for at least 12 hours. In other embodiments, compound 1, or a pharmaceutically acceptable salt thereof, and combination partners described herein, are administered to a subject who has fasted overnight.

In other embodiments, compound 1, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, compound 1, or a pharmaceutically acceptable salt thereof, is administered to a subject in a fed state 30 minutes post-meal. In other instances, compound 1, or a pharmaceutically acceptable salt thereof, and combination partners described herein, are administered to a subject in a fed state 1 hour post-meal. In yet further embodiments, compound 1, or a pharmaceutically acceptable salt thereof, is administered to a subject with food.

In some embodiments of a method of treating cancer, compound 1, or a pharmaceutically acceptable salt thereof, and combination partners described herein are administered in 28-day cycles. In some embodiments of a method of treating cancer, compound 1, or a pharmaceutically acceptable salt thereof, and combination partners described herein, are administered for multiple 28-day cycles. In some embodiments of a method of treating cancer, compound 1, or a pharmaceutically acceptable salt thereof, and combination partners described herein, are administered for at least one 28-day cycle. In some embodiments of a method of treating cancer, compound 1, or a pharmaceutically acceptable salt thereof, and combination partners described herein, are administered for at least two 28-day cycles. In some embodiments of a method of treating cancer, compound 1, or a pharmaceutically acceptable salt thereof, and combination partners described herein, are administered for at least three 28-day cycles. In some embodiments of a method of treating cancer, compound 1, or a pharmaceutically acceptable salt thereof, and combination partners described herein, are administered for at least four 28-day cycles. In some embodiments of a method of treating cancer, compound 1, or a pharmaceutically acceptable salt thereof, and combination partners described herein, are administered for at least five 28-day cycles. In some embodiments of a method of treating cancer, compound 1, or a pharmaceutically acceptable salt thereof, and combination partners described herein, are administered for at least six 28-day cycles.

In some embodiments of a method of treating cancer, compound 1, or a pharmaceutically acceptable salt thereof, is administered on days 1-7 of each 28-day cycle. In some embodiments of a method of treating cancer, compound 1, or a pharmaceutically acceptable salt thereof, is administered on days 1-14 of each 28-day cycle. In some embodiments of a method of treating cancer, compound 1, or a pharmaceutically acceptable salt thereof, is administered on days 1-21 of each 28-day cycle. In some embodiments of a method of treating cancer, compound 1, or a pharmaceutically acceptable salt thereof, is administered on days 1-28 of each 28-day cycle.

In some embodiments of a method of treating cancer, compound 1, or a pharmaceutically acceptable salt thereof, is administered twice a day on day 1 of a 28-day cycle. In some embodiments of a method of treating cancer, compound 1, or a pharmaceutically acceptable salt thereof, is administered twice a day on day 8 of a 28-day cycle. In some embodiments of a method of treating cancer, compound 1, or a pharmaceutically acceptable salt thereof, is administered twice a day on day 15 of a 28-day cycle. In some embodiments of a method of treating cancer, compound 1, or a pharmaceutically acceptable salt thereof, is administered twice a day on day 22 of a 28-day cycle. In some embodiments of a method of treating cancer, compound 1, or a pharmaceutically acceptable salt thereof, is not administered twice a day on day 22 of a 28-day cycle.

In some embodiments of a method of treating cancer, compound 1, or a pharmaceutically acceptable salt thereof, is administered twice a day on day 1, day 8, and day 15 of a 28-day cycle.

In some embodiments of a method of treating cancer, compound 1, or a pharmaceutically acceptable salt thereof, is not administered on days 2-7, days 9-14, days 16-21, days 23-28 of a 28-day cycle.

In some embodiments of a method of treating cancer, compound 1, or a pharmaceutically acceptable salt thereof, and combination partners described herein are administered in 35-day cycles. In some embodiments of a method of treating cancer, compound 1, or a pharmaceutically acceptable salt thereof, and combination partners described herein are administered for multiple 35-day cycles. In some embodiments of a method of treating cancer, compound 1, or a pharmaceutically acceptable salt thereof, and combination partners described herein are administered for at least one 35-day cycle. In some embodiments of a method of treating cancer, compound 1, or a pharmaceutically acceptable salt thereof, and combination partners described herein are administered for at least two 35-day cycle. In some embodiments of a method of treating cancer, compound 1, or a pharmaceutically acceptable salt thereof, and combination partners described herein are administered for at least three 35-day cycle. In some embodiments of a method of treating cancer, compound 1, or a pharmaceutically acceptable salt thereof, and combination partners described herein are administered for at least four 35-day cycle. In some embodiments of a method of treating cancer, compound 1, or a pharmaceutically acceptable salt thereof, and combination partners described herein are administered for at least five 35-day cycle. In some embodiments of a method of treating cancer, compound 1, or a pharmaceutically acceptable salt thereof, and combination partners described herein are administered for at least six 35-day cycle.

In some embodiments of a method of treating cancer, compound 1, or a pharmaceutically acceptable salt thereof, is administered on days 1-7 of each 35-day cycle. In some embodiments of a method of treating cancer, compound 1, or a pharmaceutically acceptable salt thereof, is administered on days 1-14 of each 35-day cycle. In some embodiments of a method of treating cancer, compound 1, or a pharmaceutically acceptable salt thereof, is administered on days 1-21 of each 35-day cycle. In some embodiments of a method of treating cancer, compound 1, or a pharmaceutically acceptable salt thereof, is administered on days 1-28 of each 35-day cycle. In some embodiments of a method of treating cancer, compound 1, or a pharmaceutically acceptable salt thereof, is administered on days 1-35 of each 35-day cycle.

In some embodiments of a method of treating cancer, compound 1, or a pharmaceutically acceptable salt thereof, is administered twice a day on day 1 of a 35-day cycle. In some embodiments of a method of treating cancer, compound 1, or a pharmaceutically acceptable salt thereof, is administered twice a day on day 8 of a 35-day cycle. In some embodiments of a method of treating cancer, compound 1, or a pharmaceutically acceptable salt thereof, is administered twice a day on day 15 of a 35-day cycle. In some embodiments of a method of treating cancer, compound 1, or a pharmaceutically acceptable salt thereof, is administered twice a day on day 22 of a 35-day cycle. In some embodiments of a method of treating cancer, compound 1, or a pharmaceutically acceptable salt thereof, is administered twice a day on day 29 of a 35-day cycle. In some embodiments of a method of treating cancer, compound 1, or a pharmaceutically acceptable salt thereof, is not administered twice a day on day 29 of a 35-day cycle.

In some embodiments of a method of treating a cancer, compound 1, or a pharmaceutically acceptable salt thereof, is administered twice a day on day 1, day 8, day 15, and day 22 of a 35-day cycle.

In some embodiments of a method of treating cancer, compound 1, or a pharmaceutically acceptable salt thereof, is not administered on days 2-7, days 9-14, days 16-21, days 23-28, and days 30-35 of a 28-day cycle.

In some embodiments of a method of treating cancer, compound 1, or a pharmaceutically acceptable salt thereof, is administered orally.

In some embodiments of a method of treating cancer, compound 1, or a pharmaceutically acceptable salt thereof, is administered in an amount that is between about 25 mg/day and about 300 mg/day.

In some embodiments of a method of treating cancer, compound 1, or a pharmaceutically acceptable salt thereof, is administered in an amount that is between about 25 mg/day and about 250 mg/day.

In some embodiments of a method of treating cancer, compound 1, or a pharmaceutically acceptable salt thereof, is administered in an amount that is between about 25 mg/day and about 150 mg/day.

In some embodiments of a method of treating cancer, compound 1, or a pharmaceutically acceptable salt thereof, is administered in an amount that is about 25 mg/day, 50 mg/day, about 75 mg/day, about 100 mg/day, about 125 mg/day, about 150 mg/day, about 175 mg/day, about 200 mg/day, about 225 mg/day, or about 250 mg/day.

In some embodiments of a method of treating cancer, compound 1, or a pharmaceutically acceptable salt thereof, is administered in an amount that is about 25 mg/day, about 50 mg/day, about 100 mg/day, or about 150 mg/day. In some embodiments of a method of treating cancer, compound 1, or a pharmaceutically acceptable salt thereof, is administered in an amount that is about 25 mg/day. In some embodiments of a method of treating cancer, compound 1, or a pharmaceutically acceptable salt thereof, is administered in an amount that is about 50 mg/day. In some embodiments of a method of treating cancer, compound 1, or a pharmaceutically acceptable salt thereof, is administered in an amount that is about 100 mg/day. In some embodiments of a method of treating cancer, compound 1, or a pharmaceutically acceptable salt thereof, is administered in an amount that is about 150 mg/day. In some embodiments, compound 1, or a pharmaceutically acceptable salt thereof, is administered in an amount that is about 250 mg/day.

In some embodiments of a method of treating cancer, compound 1, or a pharmaceutically acceptable salt thereof, is administered in an amount that is about 25 mg, 30 mg, 40 mg, 50 mg, about 60 mg, about 65 mg about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

In some embodiments of a method of treating cancer, compound 1, or a pharmaceutically acceptable salt thereof, is administered in an amount that is between about 25 mg and about 300 mg twice a day, once a week (BID-QW).

In some embodiments of a method of treating cancer, compound 1, or a pharmaceutically acceptable salt thereof, is administered in an amount that is between about 25 mg and about 250 mg twice a day, once a week (BID-QW).

In some embodiments of a method of treating cancer, compound 1, or a pharmaceutically acceptable salt thereof, is administered in an amount that is between about 25 mg and about 150 mg twice a day, once a week (BID-QW).

In some embodiments of a method of treating cancer, compound 1, or a pharmaceutically acceptable salt thereof, is administered in an amount that is about 25 mg, 50 mg, about 75 mg, about 100 mg, about 150 mg, about 175 mg, about 200 mg, about 225 mg, or about 250 mg twice a day, once a week (BID-QW).

In some embodiments of a method of treating cancer, compound 1, or a pharmaceutically acceptable salt thereof, is administered in an amount that is about 25 mg, 50 mg, about 100 mg, or about 150 mg twice a day, once a week (BID-QW).

In some embodiments of a method of treating cancer, compound 1, or a pharmaceutically acceptable salt thereof, is administered once a day (QD). In some embodiments of a method of treating cancer, compound 1, or a pharmaceutically acceptable salt thereof, is administered twice a day (BID). In some embodiments of a method of treating cancer, compound 1, or a pharmaceutically acceptable salt thereof, is administered three times a day (TID).

In some embodiments of a method of treating cancer, compound 1, or a pharmaceutically acceptable salt thereof, is administered once a week. In some embodiments of a method of treating cancer, compound 1, or a pharmaceutically acceptable salt thereof, is administered twice a week. In some embodiments of a method of treating cancer, compound 1, or a pharmaceutically acceptable salt thereof, is administered three times a week.

In some embodiments of a method of treating cancer, compound 1, or a pharmaceutically acceptable salt thereof, is administered once a day, once a week (QD-QW). In some embodiments of a method of treating cancer, compound 1, or a pharmaceutically acceptable salt thereof, is administered twice a day, once a week (BID-QW).

In some embodiments, compound 1, or a pharmaceutically acceptable salt thereof, is administered in an amount that is between about 25 mg and about 300 mg twice a day, once a week (BID-QW).

In some embodiments, compound 1, or a pharmaceutically acceptable salt thereof, is administered in an amount that is between about 25 mg and about 250 mg twice a day, once a week (BID-QW).

In some embodiments, compound 1, or a pharmaceutically acceptable salt thereof, is administered in an amount that is between about 25 mg and about 150 mg twice a day, once a week (BID-QW).

In some embodiments, compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

In some embodiments, compound 1, or a pharmaceutically acceptable salt thereof, is administered in an amount that is about 25 mg, 50 mg, about 100 mg, about 125 mg, or about 150 mg twice a day, once a week (BID-QW).

In some embodiments, compound 1, or a pharmaceutically acceptable salt thereof, is administered in an amount that is about 125 mg twice a day, once a week (BID-QW).

In some embodiments, compound 1, or a pharmaceutically acceptable salt thereof, is administered in an amount that is about 250 mg once a day, once a week.

In some embodiments, compound 1, or a pharmaceutically acceptable salt thereof, is administered in an amount that is about 25 mg, 30 mg, 40 mg, 50 mg, about 60 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 290 mg, or about 300 mg.

In some embodiments, each of the above-recited amounts may be administered QD, QW, BID, BID-QD, or BID-QW.

### Compound 2

In some embodiments, compound 2, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, compound 2, or a pharmaceutically acceptable salt thereof, is administered to a subject who is in a fasted state for at least 8 hours. In other embodiments, compound 2, or a pharmaceutically acceptable salt thereof, and combination partners described herein, are administered to a subject who is in a fasted state for at least 10 hours. In yet other embodiments, compound 2, or a pharmaceutically acceptable salt thereof, and combination partners described herein, are administered to a subject who is in a fasted state for at least 12 hours. In other embodiments, compound 2, or a pharmaceutically acceptable salt thereof, and combination partners described herein, are administered to a subject who has fasted overnight.

In other embodiments, compound 2, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, compound 2, or a pharmaceutically acceptable salt thereof, is administered to a subject in a fed state 30 minutes post-meal. In other instances, compound 2, or a pharmaceutically acceptable salt thereof, and combination partners described herein, are administered to a subject in a fed state 1 hour post-meal. In yet further embodiments, compound 2, or a pharmaceutically acceptable salt thereof, is administered to a subject with food.

In some embodiments of a method of treating cancer, compound 2, or a pharmaceutically acceptable salt thereof, and combination partners described herein are administered in 28-day cycles. In some embodiments of a method of treating cancer, compound 2, or a pharmaceutically acceptable salt thereof, and combination partners described herein, are administered for multiple 28-day cycles. In some embodiments of a method of treating cancer, compound 2, or a pharmaceutically acceptable salt thereof, and combination partners described herein, are administered for at least one 28-day cycle. In some embodiments of a method of treating cancer, compound 2, or a pharmaceutically acceptable salt thereof, and combination partners described herein, are administered for at least two 28-day cycles. In some embodiments of a method of treating cancer, compound 2, or a pharmaceutically acceptable salt thereof, and combination partners described herein, are administered for at least three 28-day cycles. In some embodiments of a method of treating cancer, compound 2, or a pharmaceutically acceptable salt thereof, and combination partners described herein, are administered for at least four 28-day cycles. In some embodiments of a method of treating cancer, compound 2, or a pharmaceutically acceptable salt thereof, and combination partners described herein, are administered for at least five 28-day cycles. In some embodiments of a method of treating cancer, compound 2, or a pharmaceutically acceptable salt thereof, and combination partners described herein, are administered for at least six 28-day cycles.

In some embodiments of a method of treating cancer, compound 2, or a pharmaceutically acceptable salt thereof, is administered on days 1-7 of each 28-day cycle. In some embodiments of a method of treating cancer, compound 2, or a pharmaceutically acceptable salt thereof, is administered on days 1-14 of each 28-day cycle. In some embodiments of a method of treating cancer, compound 2, or a pharmaceutically acceptable salt thereof, is administered on days 1-21 of each 28-day cycle. In some embodiments of a method of treating cancer, compound 2, or a pharmaceutically acceptable salt thereof, is administered on days 1-28 of each 28-day cycle.

In some embodiments of a method of treating cancer, compound 2, or a pharmaceutically acceptable salt thereof, is administered on days 1-7 of each 21-day cycle. In some embodiments of a method of treating cancer, compound 2, or a pharmaceutically acceptable salt thereof, is administered on days 1-14 of each 21-day cycle. In some embodiments of a method of treating cancer, compound 2, or a pharmaceutically acceptable salt thereof, is administered on days 1-21 of each 21-day cycle.

In some embodiments of a method of treating cancer, compound 2, or a pharmaceutically acceptable salt thereof, is administered orally.

In some embodiments of a method of treating cancer, compound 2 is administered once a day (QD). In some embodiments of a method of treating cancer, compound 2 is administered twice a day (BID).

In some embodiments of a method of treating cancer, compound 2, or a pharmaceutically acceptable salt thereof, is administered once a week. In some embodiments of a method of treating cancer, compound 2, or a pharmaceutically acceptable salt thereof, is administered twice a week. In some embodiments of a method of treating cancer, compound 2, or a pharmaceutically acceptable salt thereof, is administered on day 1 and day 2 of each week. In some embodiments of a method of treating cancer, compound 2, or a pharmaceutically acceptable salt thereof, is administered on day 1 and day 3 of each week. In some embodiments of a method of treating cancer, compound 2, or a pharmaceutically acceptable salt thereof, is administered on day 1 and day 4 of each week. In some embodiments of a method of treating cancer, compound 2, or a pharmaceutically acceptable salt thereof, is administered on day 1 and day 5 of each week.

In some embodiments of a method of treating cancer, compound 2, or a pharmaceutically acceptable salt thereof, is administered three times a week. In some embodiments of a method of treating cancer, wherein compound 2, or a pharmaceutically acceptable salt thereof, is administered on day 1, day 3, and day 5. In some embodiments of a method of treating cancer, wherein compound 2, or a pharmaceutically acceptable salt thereof, is administered on day 1, day 2, and day 3. In some embodiments of a method of treating cancer, wherein compound 2, or a pharmaceutically acceptable salt thereof, is administered on day 1, day 3, and day 4. In some embodiments of a method of treating cancer, wherein compound 2, or a pharmaceutically acceptable salt thereof, is administered on day 1, day 2, and day 4. In some embodiments of a method of treating cancer, wherein compound 2, or a pharmaceutically acceptable salt thereof, is administered on day 1, day 2, and day 5.

In some embodiments of a method of treating cancer, wherein compound 2, or a pharmaceutically acceptable salt thereof, is administered in an amount that is between about 1 mg/day and about 500 mg/day. In some embodiments of a method of treating cancer, compound 2, or a pharmaceutically acceptable salt thereof, is administered in an amount that is between about 20 mg/day and about 400 mg/day. In some embodiments of a method of treating cancer, compound 2, or a pharmaceutically acceptable salt thereof, is administered in an amount that is between about 30 mg/day and about 300 mg/day. In some embodiments of a method of treating cancer, compound 2, or a pharmaceutically acceptable salt thereof, is administered in an amount that is between about 10 mg/day and about 100 mg/day. In some embodiments of a method of treating cancer, compound 2, or a pharmaceutically acceptable salt thereof, is administered in an amount that is between about 20 mg/day and about 80 mg/day. In some embodiments of a method of treating cancer, wherein compound 2, or a pharmaceutically acceptable salt thereof, is administered in an amount that is between about 20 mg/day and about 60 mg/day.

In some embodiments of a method of treating cancer, wherein compound 2, or a pharmaceutically acceptable salt thereof, is administered in an amount that is about 20 mg/day. In some embodiments of a method of treating cancer, wherein compound 2, or a pharmaceutically acceptable salt thereof, is administered in an amount that is about 30 mg/day. In some embodiments of a method of treating cancer, wherein compound 2, or a pharmaceutically acceptable salt thereof, is administered in an amount that is about 40 mg/day. In some embodiments of a method of treating cancer, wherein compound 2, or a pharmaceutically acceptable salt thereof, is administered in an amount that is about 50 mg/day. In some embodiments of a method of treating cancer, wherein compound 2, or a pharmaceutically acceptable salt thereof, is administered in an amount that is about 60 mg/day.

In some embodiments, the dosing of compound 2, or a pharmaceutically acceptable salt thereof, is in any suitable amount to treat the cancer. For example, the dosing is a daily dosage of between 1 mg up to 500 mg. As an additional example, the daily dose is in a range from about 20 mg to 400 mg (or any sub-range or sub-value there between, including endpoints). In some embodiments, the range of dosing of compound 2, or a pharmaceutically acceptable salt thereof, is from 10 mg to 300 mg. In some embodiments, the range of dosing of compound 2, or a pharmaceutically acceptable salt thereof, is from 10 mg to 100 mg. In some embodiments, the range of dosing of compound 2, or a pharmaceutically acceptable salt thereof, is from 5 mg to 50 mg. In some embodiments, compound 2, or a pharmaceutically acceptable salt thereof, is administered in an amount that is about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg. The daily dosage can be achieved by administering a single administered dosage (e.g., QD) or via multiple administrations during a day (e.g., BID, TID, QID, etc.) to provide the total daily dosage. In some embodiments, the compound 2 is administered once a day QD. In some embodiments, the compound 2 is administered once daily (QD) for 21 days followed by a 7-day break (3 weeks on, 1 week off) on a 28-day cycle. In some embodiments, compound 2, or a pharmaceutically acceptable salt thereof, is administered twice a day (BID). In some embodiments, compound 2, or a pharmaceutically acceptable salt thereof, is administered twice a day for 21 days followed by a 7-day break (3 weeks on, 1 week off) on a 28-day cycle.

In some embodiments, compound 2, or a pharmaceutically acceptable salt thereof, is administered QD or BID for 2 weeks on and 1 week off (21 day schedule). In some embodiments, compound 2, or a pharmaceutically acceptable salt thereof, is administered QD or BID for 3 weeks on and 1 week off (28 day schedule). In some embodiments, compound 2, or a pharmaceutically acceptable salt thereof, is administered QD or BID three times a week (D1D3D5 TIW) e.g., Day 1, Day 3, and Day 5. In some embodiments, compound 2, or a pharmaceutically acceptable salt thereof, is administered twice a day/twice a week e.g., Day 1 and Day 2 (BID-D1D2-BIW).

In some embodiments, compound 2, or a pharmaceutically acceptable salt thereof, is administered once a day (QD) continuous dosing at a dose of 20 mg/day to 60 mg/day, 40 mg/day, or 60 mg/day. In some embodiments, compound 2, or a pharmaceutically acceptable salt thereof, is administered twice a day (BID) continuous dosing at a dose of 20 mg/day to 80 mg/day. In some embodiments, compound 2, or a pharmaceutically acceptable salt thereof, is administered twice a day (BID) continuous dosing at a dose of 10 mg/day to 100 mg/day.

### Ruxolitinib

In some embodiments of a method of treating cancer, ruxolitinib is administered orally. In some embodiments of a method of treating cancer, ruxolitinib is administered twice a day (BID). In some embodiments of a method of treating cancer, ruxolitinib is administered in an amount that is between about 20 mg/day and 100 mg/day. In some embodiments of a method of treating cancer, ruxolitinib is administered in an amount that is about 15 mg/day. In some embodiments of a method of treating cancer, ruxolitinib is administered in an amount that is about 20 mg/day. In some embodiments of a method of treating cancer, ruxolitinib is administered in an amount that is about 30 mg/day. In some embodiments of a method of treating cancer, ruxolitinib is administered in an amount that is about 40 mg/day. In some embodiments of a method of treating cancer, ruxolitinib is administered in an amount that is about 50 mg/day. In some embodiments of a method of treating cancer, ruxolitinib is administered in an amount that is about 60 mg/day. In some embodiments of a method of treating cancer, ruxolitinib is administered in an amount that is about 70 mg/day. In some embodiments of a method of treating cancer, ruxolitinib is administered in an amount that is about 80 mg/day. In some embodiments of a method of treating cancer, ruxolitinib is administered in an amount that is about 90 mg/day. In some embodiments of a method of treating cancer, ruxolitinib is administered in an amount that is about 100 mg/day.

### EXAMPLES

### Experimental Procedure:

In vivo experiments will require breeding of conditional JAK2V617F knock-in mice with Mx1Cre transgenic mice. Appropriate experimental mice will be identified by genotyping. The JAK2V617F expression will be induced in mice bone marrow by intraperitoneal injection of pI-pC. At 6-8 weeks after induction of JAK2V617F expression, bone marrow cells will be transplanted into lethally irradiated syngeneic C57BL/6 mice to generate a cohort of mice. At 6 weeks after transplantation, blood counts will be measured to confirm MPN disease development. Mice will be randomized to receive treatment with vehicle, compound 2 alone or in combination with Ruxolitinib.

### Example 1

Determine the effects of compound 2/compound 1 alone or in combination with Ruxolitinib on proliferation and apoptosis of a panel of JAK2V617F-positive leukemia cell lines (e.g., BA/F3-EpoR-JAK2V617F, HEL, SET2, UKE-1). Cells will be treated with various concentrations of compound 2/compound 1 alone or in combination with a fixed concentration (IC₅₀) of Ruxolitinib. Cell viability/proliferation will be determined by cell titer glow and apoptosis will be assessed by Annexin V staining and flow cytometry.

### Example 2

Test the effects of compound 2/compound 1 alone or in combination with Ruxolitinib on hematopoietic progenitors from JAK2V617F knock-in mice and MPN patients using hematopoietic progenitor colony assays.

### Example 3

Determine the mechanism of action of compound 2/Ruxolitinib combination on JAK2 mutant cell lines. Western blotting will be performed on cell lysates from HEL and UKE-1 cells treated with DMSO, compound 2, Ruxolitinib or compound 2/Ruxolitinib combination to assess the status of phospho-SHP2, ERK, AKT and STAT5/STAT3 signaling.

### Example 4

Test the in vivo efficacy of compound 2 alone or in combination with Ruxolitinib in JAK2V617F knock-in mouse model of MPN. Mice will be monitored by periodic blood counts. At first, different doses of compound 2 alone will be administered in a small cohort of animals to identify the suitable dose for in vivo studies. Peripheral blood counts and histopathological analysis will be performed to determine the efficacy and tolerability of compound 2. The right dose of compound 2 will be combined with Ruxolitinib to determine the combinatorial effects in MPN mouse model. Ruxolitinib will be administered at 60 mg/kg per day by oral gavage. At the end of study, mice will be analyzed by blood counts, flow cytometry, hematopoietic progenitor colony assays and histopathology.

The Examples below demonstrate the combination of Compound 2 and ruxolitinib provides a combination benefit in JAK2V617F-driven MPN preclinical models.

### Example 5: The Combination of Compound 2+Ruxolitinib Demonstrated a Robust Inhibition of Cell Viability in JAK2V617F Mutant UKE-1 Cells

### Method and Results:

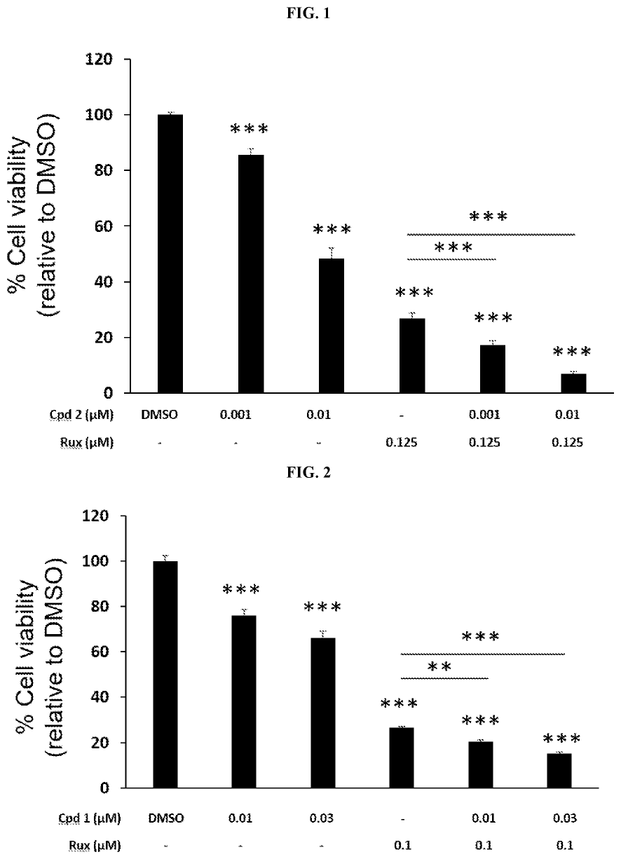

Human JAK2V617F-positive UKE-1 cells were treated with vehicle (DMSO), Compound 2, ruxolitinib, or Compound 2+ruxolitinib for 6 days. Medium was changed every two days with the addition of fresh drug. Cell viability was assessed by CellTiter-Glo. Data are represented in bar graphs as mean±SEM (***p<0.001) in FIG. 1. As shown in FIG. 1, the combined treatment of Compound 2 and ruxolitinib resulted in significantly greater inhibition of UKE-1 cells compared to single drug treatments. Thus, this data demonstrates that Compound 2 with ruxolitinib provides an in vitro combination benefit in JAK2V617F mutant UKE-1 cells.

### Example 6: The Combination of Compound 2+Ruxolitinib Demonstrated a Robust Inhibition of Cell Viability in JAK2V617F Mutant HEL Cells

### Method and Results:

Human JAK2V617F-positive HEL cells were treated with vehicle (DMSO), Compound 2, ruxolitinib, or Compound 2 plus ruxolitinib for 6 days. Medium was changed every two days with the addition of fresh drugs. Cell viability was assessed by CellTiter-Glo. Data are represented in bar graphs as mean±SEM (**p<0.005; ***p<0.001) in FIG. 3. As shown in FIG. 3, the combined treatment of Compound 2 and ruxolitinib resulted in significantly greater inhibition of HEL cells compared to single drug treatments. Thus, this data demonstrates that Compound 2 with ruxolitinib provides in vitro combination benefit in JAK2V617F mutant HEL cells.

### Example 7: The Combination of Compound 2+Ruxolitinib Induced Apoptosis in JAK2V617F Mutant UKE-1 Cells

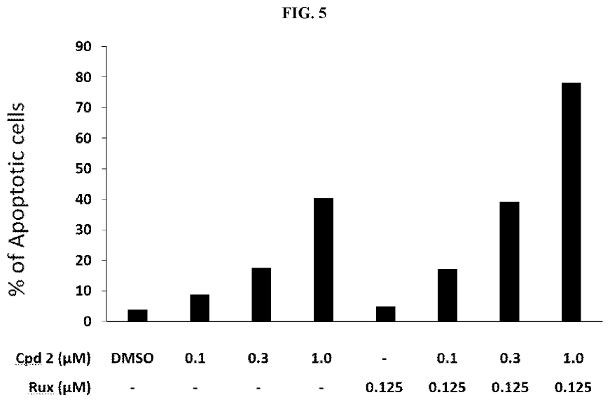

The following experiment was designed to test whether the combination of Compound 2 and ruxolitinib induces apoptosis in UKE-1 cells harboring a JAK2V617F mutation. Human JAK2V617F-positive UKE-1 cells were treated with vehicle (DMSO), Compound 2, ruxolitinib, or Compound 2 plus ruxolitinib for 72 hours. Cell apoptosis was determined by Annexin V staining and flow cytometry. FIG. 5 demonstrates that the treatment of Compound 2 induced apoptosis in UKE-1 cells, and combined treatment of Compound 2 and ruxolitinib resulted a robust apoptosis in UKE-1 cells compared to single drug treatments. Thus, the combination of Compound 2 and ruxolitinib induced robust apoptosis in UKE-1 cells harboring a JAK2V617F mutation.

### Example 8: Compound 2+Ruxolitinib Demonstrated In Vivo Combination Benefit by Reducing Splenomegaly in a Jak2V617F Knock-In Mouse Model of MPN

### Method and Results:

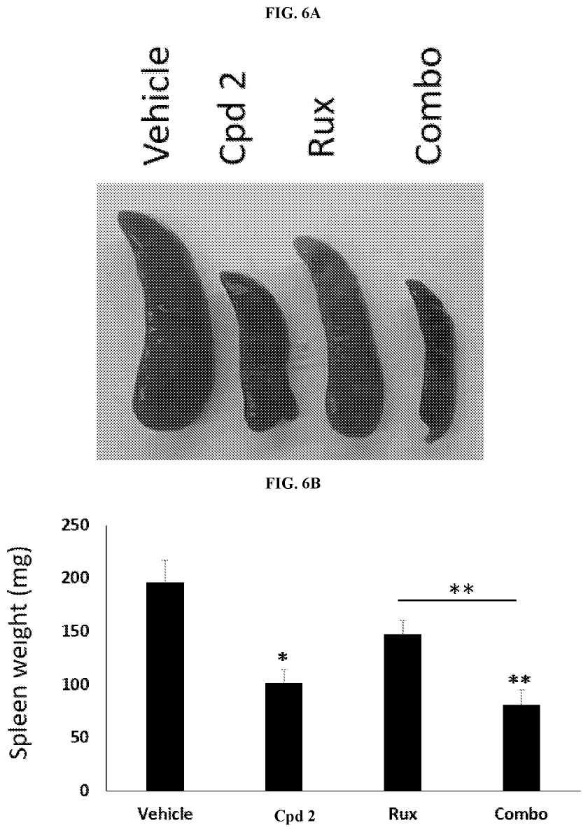

In animal models, JAK2V617F expression typically leads to the development of MPN-like disease which results in enlargement of spleen (splenomegaly), higher cell counts, and fibrotic evolution. JAK2V617F knock-in mouse models were used to investigate the effect of the combination of Compound 2 with ruxolitinib. Animals were dosed with either Compound 2, ruxolitinib, or the combination of Compound 2 with ruxolitinib. As shown in FIGS. 6A and 6B, the spleen size/weight was significantly reduced in Jak2V617F mice by Compound 2 (30 mg/kg/per day) treatment compared with vehicle treatment while combined treatment of Compound 2 (30 mg/kg/per day) and ruxolitinib (60 mg/kg/per day) resulted in an even greater reduction of spleen size compared to ruxolitinib treatment. Data are shown in bar graphs as mean±SEM (n=4 for vehicle; n=7 for Compound 2 I; n=4 for ruxolitinib; n=7 for combo drug treatment) in FIG. 6B. (*p<0.05; **p<0.005). Overall, the data demonstrates that the combination of Compound 2 and ruxolitinib resulted in an in vivo combination benefit in JAK2V617F knock-in mice.

### Example 9: The Combination of Compound 2 and Ruxolitinib Reduced Hematopoietic Progenitor Colonies in MPN Patient CD34+ Cells Ex Vivo

### Method and Results:

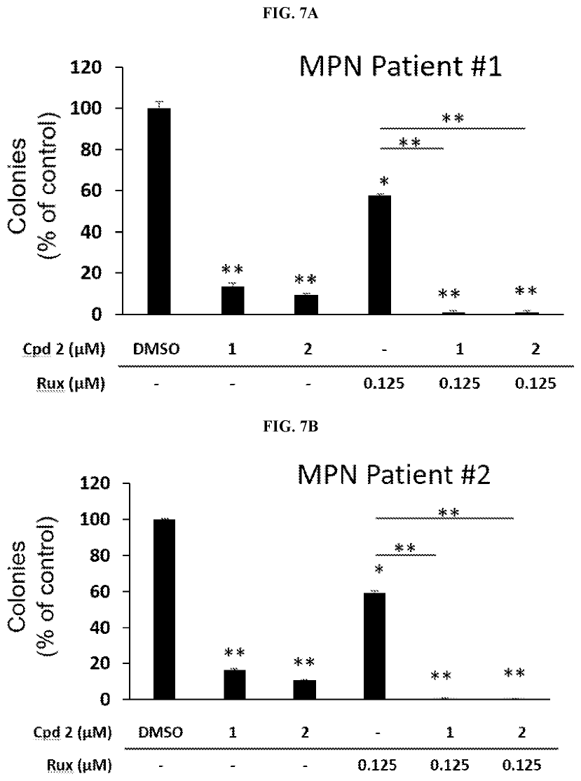

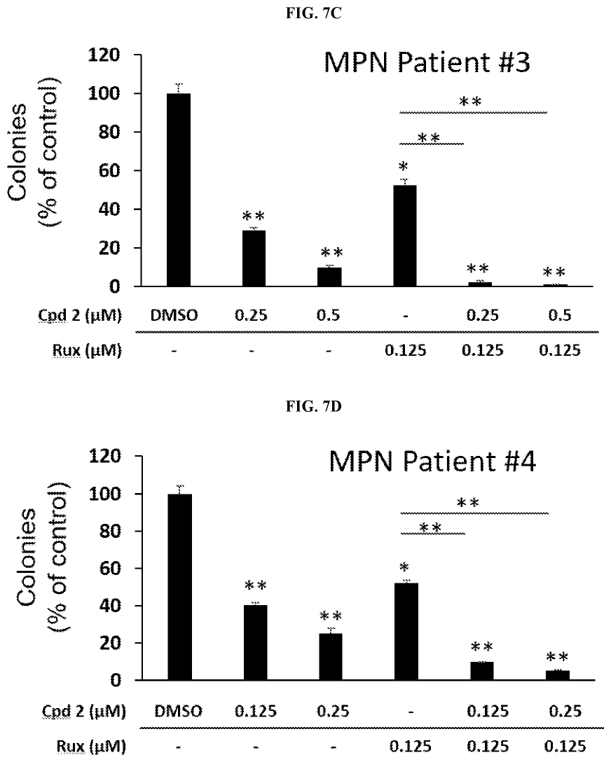

CD34+ cells were isolated from the peripheral blood of MPN patients (PV and MF). CD34+ cells were plated in methylcellulose medium containing cytokines in the presence of vehicle (DMSO), Compound 2, ruxolitinib, or Compound 2 in combination with ruxolitinib. Hematopoietic progenitor colonies (CFU-GM and BFU-E) were counted after 14 days. As shown in FIGS. 7A-7D, the combination of Compound 2 and ruxolitinib significantly reduced hematopoietic progenitor colonies from four distinct MPN patient CD34+ cells ex vivo when compared to single-drug treatments. (*p<0.05; **p<0.005). Thus, the combination of Compound 2 and ruxolitinib demonstrated ex vivo combination benefit in CD34+ cells from four distinct patients.

The Examples below demonstrate the combination of Compound 1 and ruxolitinib provides a combination benefit in JAK2V617F-driven MPN preclinical models

### Example 10: The Combination of Compound 1+Ruxolitinib Demonstrated a Robust Inhibition of Cell Viability in JAK2V617F Mutant UKE-1 Cells

### Method and Results:

Human JAK2 V617F-positive UKE-1 cells were treated with vehicle (DMSO), Compound 1, ruxolitinib, or Compound 1plus ruxolitinib for 6 days. Medium was changed every two days with the addition of fresh drugs. Cell viability was assessed by CellTiter-Glo. Data are represented in bar graphs as mean±SEM (***p<0.001) in FIG. 2. As shown in FIG. 2, the combined treatment of Compound 1 and ruxolitinib resulted in significantly greater inhibition of UKE-1 cells compared to single drug treatments. The results indicate that the combination Compound land ruxolitinib demonstrated an in vitro combination benefit.

### Example 11: The Combination of Compound 1+Ruxolitinib Demonstrated a Robust Inhibition of Cell Viability in JAK2V617F Mutant HEL Cells

### Material and Methods:

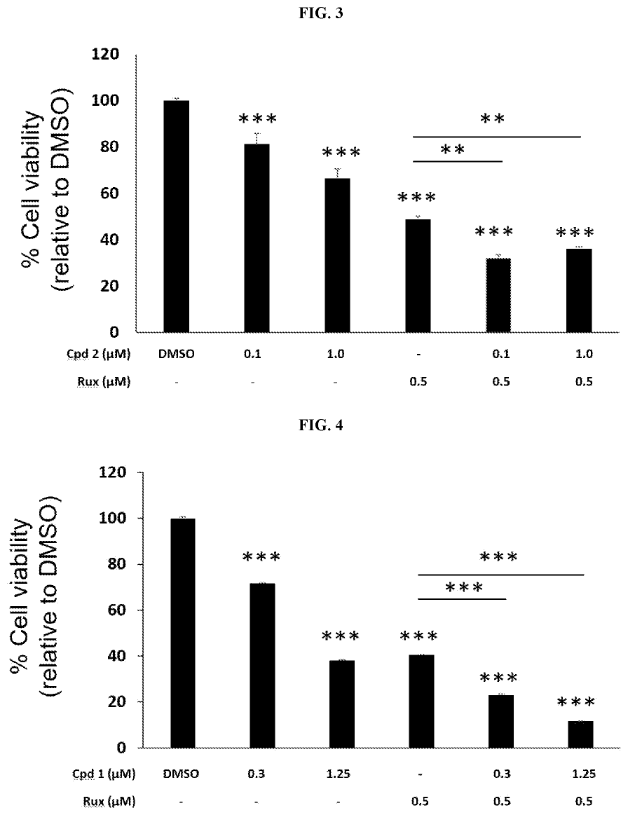

Human JAK2V617F-positive HEL cells were treated with vehicle (DMSO), Compound 1, ruxolitinib, or Compound 1 plus ruxolitinib for 6 days. Medium was changed every two days with the addition of fresh drugs. Cell viability was assessed by CellTiter-Glo. Data are represented in bar graphs as mean±SEM (**p<0.005; ***p<0.001) in FIG. 4. As shown in FIG. 4, the combined treatment of Compound land ruxolitinib resulted in significantly greater inhibition of HEL cells compared to single drug treatments. The results indicate that the combination Compound land ruxolitinib demonstrated an in vitro combination benefit.

### Example 12 the Combination of Compound 1+Ruxolitinib Reduced Hematopoietic Progenitor Colonies in MPN Patient CD34+ Cells Ex Vivo

### Method and Results:

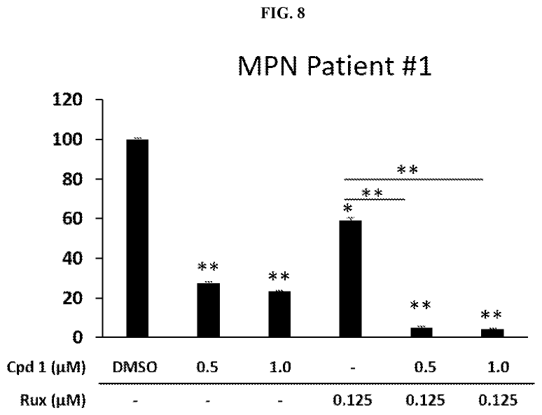

CD34+ cells were isolated from the peripheral blood of MPN patients (PV and MF). CD34+ cells were plated in methylcellulose medium containing cytokines in the presence of vehicle (DMSO), Compound 1, ruxolitinib or Compound 1 plus ruxolitinib. Hematopoietic progenitor colonies (CFU-GM and BFU-E) were counted after 14 days. As shown in FIG. 8, the results show that the combination of Compound 1 and ruxolitinib demonstrated ex vivo combination benefit in patient CD34+ cells. (*p<0.05; * *p<0.005).

1. A method of treating cancer in a subject in need thereof, the method comprising: administering to the subject in need thereof a therapeutically effective amount of
(i) compound 1: 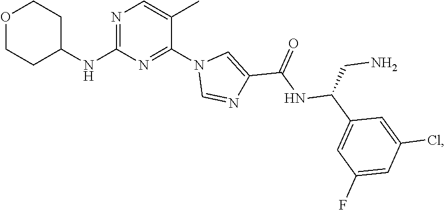 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) a JAK inhibitor.

2. The method of claim 1, further comprising a SHP2 inhibitor.

3. A method of treating cancer in a subject in need thereof, the method comprising: administering to the subject in need thereof a therapeutically effective amount of
(i) compound 2: 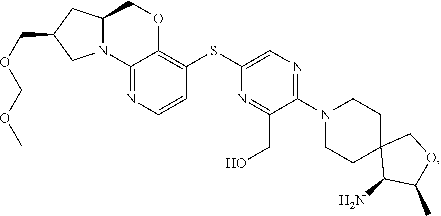 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(ii) a JAK inhibitor.

4. The method of claim 3, further comprising a ERK1/2 inhibitor.

5. The method of claim 3 or 4, further comprising compound 1: 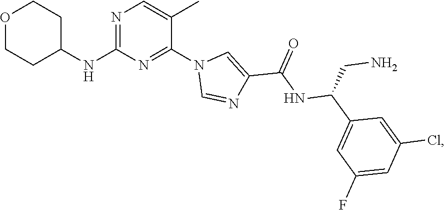 [SMILES: Cc1cnc(NC2CCOCC2)nc1-n1cnc(C(=O)N[C@H](CN)c2cc(F)cc(Cl)c2)c1]
or a pharmaceutically acceptable salt thereof.

6. A method of treating cancer in a subject in need thereof, the method comprising: administering to the subject in need thereof a therapeutically effective amount of
(i) compound 2: 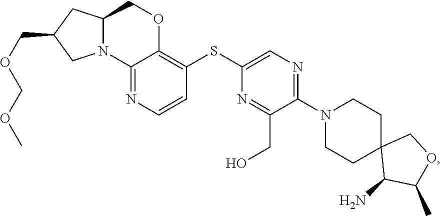 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof;
(ii) an ERK1/2 inhibitor; and
(iii) a JAK inhibitor.

7. A method of treating cancer in a subject in need thereof, the method comprising: administering to the subject in need thereof a therapeutically effective amount of
(i) compound 1: 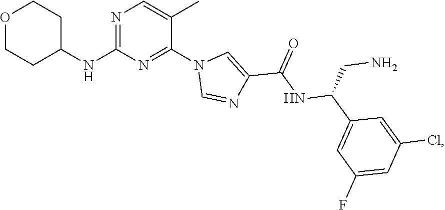 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) a SHP2 inhibitor; and
(iii) a JAK inhibitor.

8. The method of claim 2 or 7, wherein the SHP2 inhibitor is sodium stibogluconate, RMC-4550, NSC87877, SPI-112, TN0155, IACS-13909, SHP099 HCl, or compound 2: 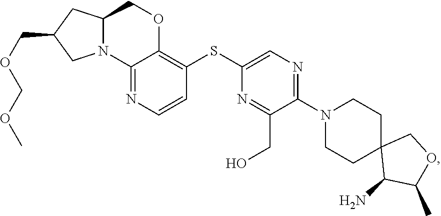 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof.

9. A method of treating cancer in a subject in need thereof, the method comprising: administering to the subject in need thereof a therapeutically effective amount of
(i) compound 1: 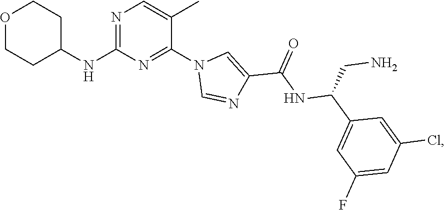 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 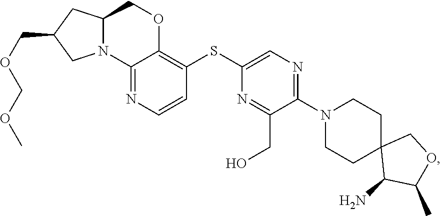 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) a JAK inhibitor.

10. The method of any one of claims 1-9, wherein the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

11. The method of any one of claims 1-10, wherein the JAK inhibitor is ruxolitinib.

12. A method of treating cancer in a subject in need thereof, the method comprising: administering to the subject in need thereof a therapeutically effective amount of
(i) compound 1: 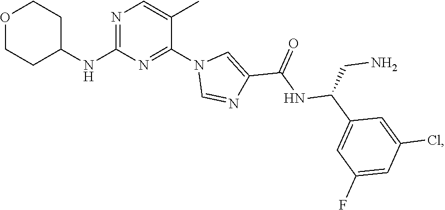 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) ruxolitinib.

13. A method of treating cancer in a subject in need thereof, the method comprising: administering to the subject in need thereof a therapeutically effective amount of
(i) compound 2: 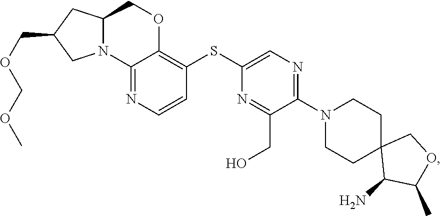 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(ii) ruxolitinib.

14. A method of treating cancer in a subject in need thereof, the method comprising: administering to the subject in need thereof a therapeutically effective amount of
(i) compound 1: 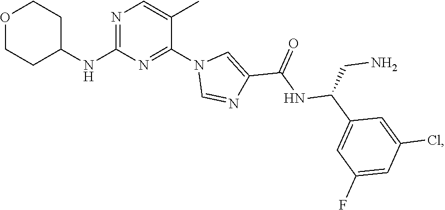 [SMILES: Cc1cnc(NC2CCOCC2)nc1-n1cnc(C(=O)N[C@H](CN)c2cc(F)cc(Cl)c2)c1]
or a pharmaceutically acceptable salt thereof;
(ii) compound 2: 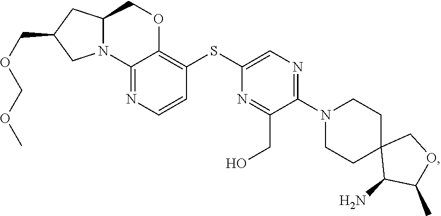 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(iii) ruxolitinib.

15. The method of any one of claims 1-14, wherein the cancer is a mitogen-activated protein kinase (MAPK) pathway driven cancer.

16. The method of any one of claims 1-14, wherein the cancer is a BRAF-driven cancer, HRAS-driven cancer, or a NRAS-driven cancer.

17. The method of any one of claims 1-14, wherein the cancer comprises at least one cancer cell driven by deregulated ERK.

18. The method of any one of claims 1-14, wherein the cancer has at least one mutation in RAS.

19. The method of any one of claims 1-14, wherein the cancer has at least one mutation in RAF.

20. The method of any one of claims 1-14, wherein the cancer has at least one mutation in MEK.

21. The method of any one of claims 1-14, wherein the cancer has a G12C KRAS mutation.

22. The method of any one of claims 1-14, wherein the cancer has a G12D KRAS mutation.

23. The method of any one of claims 1-14, wherein the cancer has a G12S KRAS mutation.

24. The method of any one of claims 1-14, wherein the cancer has a G12V KRAS mutation.

25. The method of any one of claims 1-14, wherein the cancer has a G13D KRAS mutation.

26. The method of any one of claims 1-14, wherein the cancer has a Q16H KRAS mutation.

27. The method of any one of claims 1-14, wherein the cancer has a Q16K KRAS mutation.

28. The method of any one of claims 1-14, wherein the cancer has a Q61R NRAS mutation.

29. The method of any one of claims 1-14, wherein the cancer is a BRAF V600E or V600K mutant tumor.

30. The method of any one of claims 1-14, wherein the cancer has a mutation in JAK2, CALR, MPL, or combinations thereof.

31. The method of any one of claims 1-14, wherein the cancer has a chromosomal translocation producing a BCR/ABL fusion gene.

32. The method of any one of claims 1-14, wherein the cancer has a Jak2V617F mutation.

33. The method of any one of claims 1-14, wherein the cancer is a MAPKm/MAPKi-naïve pan cancer.

34. The method of any one of claims 1-14, wherein the cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

35. The method of any one of claims 1-14, wherein the cancer comprises one or more EGFR mutation selected from the group consisting of L858R, exon 19 deletion, and T790M.

36. The method of any one of claims 1-35, wherein the cancer is a liquid tumor.

37. The method of any one of claims 1-35, wherein the cancer is a solid tumor.

38. The method of any one of claims 1-35, wherein the cancer is acute lymphoblastic leukemia (ALL), acute myeloid leukemia (AML), bladder cancer, bone cancer, breast cancer, chronic lymphocytic leukemia (CLL), chronic myeloid leukemia (CML), colorectal cancer (CRC), diffuse large b-cell lymphoma, endometrial cancer, eosinophilic myeloid neoplasm, esophageal cancer, essential thrombocythemia (ET), fallopian tube cancer, glioblastoma, glioma, head and neck cancer, hematologic neoplasm, hematopoietic neoplasm, Hodgkin's lymphoma, inflammatory breast cancer (IBC), leukemia, lung cancer, lymphoma, melanoma, multiple myeloma, myelofibrosis (MF), myelomonocytic leukemia, myeloproliferative disorder, a myeloproliferative neoplasm (MPN), NK-cell lymphoma, non-Hodgkin's lymphoma, non-small cell lung carcinoma (NSCLC), ovarian cancer, pancreatic cancer, polycythemia vera (PV), prostate cancer, salivary gland tumor, squamous cell carcinoma, T-cell leukemia, T-cell lymphoma, thyroid cancer, or urothelial carcinoma.

39. The method of any one of claims 1-35, wherein the cancer is a hematologic cancer.

40. The method of claim 39, wherein the hematologic cancer is acute lymphoblastic leukemia (ALL), acute myeloid leukemia (AML), chronic lymphocytic leukemia (CLL), chronic myeloid leukemia (CML), diffuse large b-cell lymphoma, hematologic neoplasm, hematopoietic neoplasm, Hodgkin's lymphoma, leukemia, lymphoma, multiple myeloma, myelofibrosis (MF), myelomonocytic leukemia, myeloproliferative disorder, a myeloproliferative neoplasm (MPN), NK-cell lymphoma, non-Hodgkin's lymphoma, polycythemia vera (PV), T-cell leukemia, or T-cell lymphoma.

41. The method of claim 39, wherein the hematologic cancer is leukemia, lymphoma, or multiple myeloma.

42. The method of claim 39, wherein the hematologic cancer is a myeloproliferative neoplasm (MPN).

43. The method of claim 42, wherein the myeloproliferative neoplasm is chronic myelogenous leukemia (CML), polycythemia vera (PV), primary myelofibrosis, essential thrombocythemia, chronic neutrophilic leukemia, or chronic eosinophilic leukemia.

44. The method of claim 39, wherein the hematologic cancer is myelofibrosis.

45. The method of claim 39, wherein the hematologic cancer is intermediate or high-risk myelofibrosis.

46. The method of claim 45, wherein the intermediate or high-risk myelofibrosis comprises primary myelofibrosis, post-polycythemia vera myelofibrosis, or post-essential thrombocythemia myelofibrosis.

47. A method of treating a hematologic cancer in a subject in need thereof, the method comprising: administering to the subject in need thereof a therapeutically effective amount of compound 1: 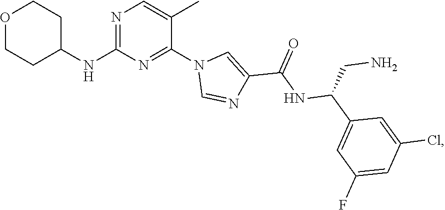 [SMILES: Cc1cnc(NC2CCOCC2)nc1-n1cnc(C(=O)N[C@H](CN)c2cc(F)cc(Cl)c2)c1]
or a pharmaceutically acceptable salt thereof.

48. A method of treating a hematologic cancer in a subject in need thereof, the method comprising: administering to the subject in need thereof a therapeutically effective amount of compound 2: 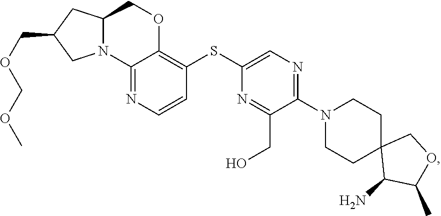 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof.

49. The method of claim 44 or 45, further comprising a JAK inhibitor.

50. A method of treating a hematologic cancer in a subject in need thereof, the method comprising: administering to the subject in need thereof a therapeutically effective amount of
(i) compound 2: 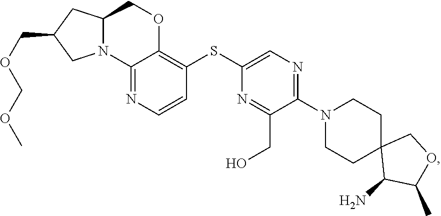 [SMILES: COCOC[C@H]1C[C@H]2COc3c(Sc4cnc(N5CCC6(CC5)CO[C@@H](C)[C@H]6N)c(CO)n4)ccnc3N2C1]
or a pharmaceutically acceptable salt thereof; and
(ii) a JAK inhibitor.

51. A method of treating a hematologic cancer in a subject in need thereof, the method comprising: administering to the subject in need thereof a therapeutically effective amount of
(i) compound 1: 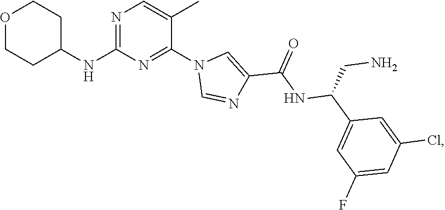 [SMILES: Cc1cnc(NC2CCOCC2)nc1-n1cnc(C(=O)N[C@H](CN)c2cc(F)cc(Cl)c2)c1]
or a pharmaceutically acceptable salt thereof; and
(ii) a JAK inhibitor.

52. The method of any one of claims 46-48, wherein the JAK inhibitor is abrocitinib, baricitinib, cerdulatinib, CHZ868, cucurbitacin I, delgocitinib, deucravacitinib, fedratinib, filgotinib, gandotinib, lestaurtinib, momelotinib, oclacitinib, pacritinib, peficitinib, ruxolitinib, tofacitinib, or upadacitinib.

53. The method of any one of claims 46-49, wherein the JAK inhibitor is ruxolitinib.

54. The method of any one of the preceding claims, wherein the pharmaceutically acceptable salt of compound 1 is the mandelic acid salt.

55. The method of any one of the preceding claims, wherein compound 1, or a pharmaceutically acceptable salt thereof, is administered orally.

56. The method of any one of the preceding claims, or a pharmaceutically acceptable salt thereof, is administered for at least one 28-day cycle.

57. The method of any one of the preceding claims, wherein compound 1, or a pharmaceutically acceptable salt thereof, is administered twice a day on day 1, day 8, day 15, and day 22 of a 28-day cycle.

58. The method of any one of the preceding claims, wherein compound 1, or a pharmaceutically acceptable salt thereof, is administered twice a day on day 1, day 8, day 15 of a 28-day cycle.

59. The method of any one of the preceding claims, wherein compound 1, or a pharmaceutically acceptable salt thereof, is not administered on days 2-7, days 9-14, days 16-21, and days 23-28 of a 28-day cycle.

60. The method of any one of the preceding claims, wherein compound 1, or a pharmaceutically acceptable salt thereof, is administered for at least one 35-day cycle.

61. The method of claim 57, wherein compound 1, or a pharmaceutically acceptable salt thereof, is administered twice a day on day 1, day 8, day 15, and day 22 of a 35-day cycle.

62. The method of any one of the preceding claims, wherein compound 1, or a pharmaceutically acceptable salt thereof, is not administered on days 2-7, days 9-14, days 16-21, days 23-28, and days 30-35 of a 28-day cycle.

63. The method of any one of the preceding claims, wherein compound 1, or a pharmaceutically acceptable salt thereof, is administered in an amount that is between about 25 mg/day and about 300 mg/day.

64. The method of any one of the preceding claims, wherein compound 1, or a pharmaceutically acceptable salt thereof, is administered in an amount that is between about 25 mg/day and about 250 mg/day.

65. The method of any one of the preceding claims, wherein compound 1, or a pharmaceutically acceptable salt thereof, is administered in an amount that is between about 25 mg/day and about 150 mg/day.

66. The method of any one of the preceding claims, wherein compound 1, or a pharmaceutically acceptable salt thereof, is administered in an amount that is about 25 mg/day, 50 mg/day, about 75 mg/day, about 100 mg/day, about 150 mg/day, about 175 mg/day, about 200 mg/day, about 225 mg/day, or about 250 mg/day.

67. The method of any one of the preceding claims, wherein compound 1, or a pharmaceutically acceptable salt thereof, is administered in an amount that is about 25 mg/day, 50 mg/day, about 100 mg/day, or about 150 mg/day.

68. The method of any one of the preceding claims, wherein compound 1, or a pharmaceutically acceptable salt thereof, is administered in an amount that is between about 25 mg and about 300 mg twice a day, once a week (BID-QW).

69. The method of any one of the preceding claims, wherein compound 1, or a pharmaceutically acceptable salt thereof, is administered in an amount that is between about 25 mg and about 250 mg twice a day, once a week (BID-QW).

70. The method of any one of the preceding claims, wherein compound 1, or a pharmaceutically acceptable salt thereof, is administered in an amount that is between about 25 mg and about 150 mg twice a day, once a week (BID-QW).

71. The method of any one of the preceding claims, wherein compound 1, or a pharmaceutically acceptable salt thereof, is administered in an amount that is about 25 mg, 50 mg, about 75 mg, about 100 mg, about 125 mg, about 150 mg, about 175 mg, about 200 mg, about 225 mg, or about 250 mg twice a day, once a week (BID-QW).

72. The method of any one of the preceding claims, wherein compound 1, or a pharmaceutically acceptable salt thereof, is administered in an amount that is about 25 mg, 50 mg, about 100 mg, or about 150 mg twice a day, once a week (BID-QW).

73. The method of any one of the preceding claims, wherein compound 1, or a pharmaceutically acceptable salt thereof, is administered in an amount that is about 125 mg twice a day, once a week (BID-QW).

74. The method of any one of the preceding claims, wherein compound 1, or a pharmaceutically acceptable salt thereof, is administered once a day (QD).

75. The method of any one of the preceding claims, wherein compound 1, or a pharmaceutically acceptable salt thereof, is administered twice a day (BID).

76. The method of any one of the preceding claims, wherein compound 1, or a pharmaceutically acceptable salt thereof, is administered three times a day (TID).

77. The method of any one of the preceding claims, wherein compound 1, or a pharmaceutically acceptable salt thereof, is administered once a week.

78. The method of any one of the preceding claims, wherein compound 1, or a pharmaceutically acceptable salt thereof, is administered twice a week.

79. The method of any one of the preceding claims, wherein compound 1, or a pharmaceutically acceptable salt thereof, is administered for at least one 28-day cycle.

80. The method of any one of the preceding claims, wherein compound 1, or a pharmaceutically acceptable salt thereof, is administered on day 1, day 8, day 15, and day 22 of a 28-day cycle.

81. The method of any one of the preceding claims, wherein compound 1, or a pharmaceutically acceptable salt thereof, is administered on day 1, day 8, day 15 of a 28-day cycle.

82. The method of any one of the preceding claims, wherein compound 2, or a pharmaceutically acceptable salt thereof, is administered orally.

83. The method of any one of the preceding claims, wherein compound 2 is administered once a day (QD).

84. The method of any one of the preceding claims, wherein compound 2 is administered twice a day (BID).

85. The method of any one of the preceding claims, wherein compound 2, or a pharmaceutically acceptable salt thereof, is administered for at least one 21-day cycle.

86. The method of claim 81, wherein compound 2, or a pharmaceutically acceptable salt thereof, is administered on days 1-14 of the 21-day cycle.

87. The method of any one of the preceding claims, wherein compound 2, or a pharmaceutically acceptable salt thereof, is administered for at least one 28-day cycle.

88. The method of claim 83, wherein compound 2, or a pharmaceutically acceptable salt thereof, is administered on days 1-21 of the 28-day cycle.

89. The method of any one of the preceding claims, wherein compound 2, or a pharmaceutically acceptable salt thereof, is administered once a week.

90. The method of any one of the preceding claims, wherein compound 2, or a pharmaceutically acceptable salt thereof, is administered twice a week.

91. The method of claim 90, wherein compound 2, or a pharmaceutically acceptable salt thereof, is administered on day 1 and day 2.

92. The method of any one of the preceding claims, wherein compound 2, or a pharmaceutically acceptable salt thereof, is administered three times a week.

93. The method of claim 92, wherein compound 2, or a pharmaceutically acceptable salt thereof, is administered on day 1, day 3, and day 5.

94. The method of any one of the preceding claims, wherein compound 2, or a pharmaceutically acceptable salt thereof, is administered in an amount that is between about 1 mg/day and about 500 mg/day.

95. The method of any one of the preceding claims, wherein compound 2, or a pharmaceutically acceptable salt thereof, is administered in an amount that is between about 20 mg/day and about 400 mg/day.

96. The method of any one of the preceding claims, wherein compound 2, or a pharmaceutically acceptable salt thereof, is administered in an amount that is between about 30 mg/day and about 300 mg/day.

97. The method of any one of the preceding claims, wherein compound 2, or a pharmaceutically acceptable salt thereof, is administered in an amount that is between about 10 mg/day and about 100 mg/day.

98. The method of any one of the preceding claims, wherein compound 2, or a pharmaceutically acceptable salt thereof, is administered in an amount that is between about 20 mg/day and about 80 mg/day.

99. The method of any one of the preceding claims, wherein compound 2, or a pharmaceutically acceptable salt thereof, is administered in an amount that is between about 20 mg/day and about 60 mg/day.

100. The method of any one of the preceding claims, wherein ruxolitinib is administered orally.

101. The method of any one of the preceding claims, wherein ruxolitinib is administered twice a day (BID).

102. The method of any one of the preceding claims, wherein ruxolitinib is administered in an amount that is about 15 mg.

103. The method of any one of the preceding claims, wherein ruxolitinib is administered in an amount that is about 20 mg.

104. The method of any one of claims 47-103, wherein the hematologic cancer is a mitogen-activated protein kinase (MAPK) pathway driven cancer.

105. The method of any one of claims 47-104, wherein the hematologic cancer is a BRAF-driven cancer, HRAS-driven cancer, or a NRAS-driven cancer.

106. The method of any one of claims 47-104, wherein the hematologic cancer comprises at least one cancer cell driven by deregulated ERK.

107. The method of any one of claims 47-104, wherein the hematologic cancer has at least one mutation in RAS.

108. The method of any one of claims 47-104, wherein the hematologic cancer has at least one mutation in RAF.

109. The method of any one of claims 47-104, wherein the hematologic cancer has at least one mutation in MEK.

110. The method of any one of claims 47-104, wherein the hematologic cancer has a G12C KRAS mutation.

111. The method of any one of claims 47-104, wherein the hematologic cancer has a G12D KRAS mutation.

112. The method of any one of claims 47-104, wherein the hematologic cancer has a G12S KRAS mutation.

113. The method of any one of claims 47-104, wherein the hematologic cancer has a G12V KRAS mutation.

114. The method of any one of claims 47-104, wherein the hematologic cancer has a G13D KRAS mutation.

115. The method of any one of claims 47-104, wherein the hematologic cancer has a Q16H KRAS mutation.

116. The method of any one of claims 47-104, wherein the hematologic cancer has a Q16K KRAS mutation.

117. The method of any one of claims 47-104, wherein the hematologic cancer has a Q61R NRAS mutation.

118. The method of any one of claims 47-104, wherein the hematologic cancer is a BRAF V600E or V600K mutant tumor.

119. The method of any one of claims 47-104, wherein the hematologic cancer is a MAPKm/MAPKi-naive pan cancer.

120. The method of any one of claims 47-104, wherein the hematologic cancer comprises one or more EGFR mutation selected from the group consisting of EGFR gene copy gain, EGFR gene amplification, chromosome 7 polysomy, L858R, exon 19 deletions/insertions, L861Q, G719C, G719S, G719A, V765A, T783A, exon 20 insertions, EGFR splice variants (Viii, Vvi, and Vii), A289D, A289T, A289V, G598A, G598V, T790M, and C797S.

121. The method of any one of claims 47-104, wherein the hematologic cancer comprises one or more EGFR mutation selected from the group consisting of L858R, exon 19 deletion, and T790M.

122. The method of any one of claims 47-121, wherein the hematologic cancer is acute lymphoblastic leukemia (ALL), acute myeloid leukemia (AML), chronic lymphocytic leukemia (CLL), chronic myeloid leukemia (CML), diffuse large b-cell lymphoma, hematologic neoplasm, hematopoietic neoplasm, Hodgkin's lymphoma, leukemia, lymphoma, multiple myeloma, myelofibrosis (MF), myelomonocytic leukemia, myeloproliferative disorder, a myeloproliferative neoplasm (MPN), NK-cell lymphoma, non-Hodgkin's lymphoma, polycythemia vera (PV), T-cell leukemia, or T-cell lymphoma.

123. The method of any one of claims 47-121, wherein the hematologic cancer is leukemia, lymphoma, or multiple myeloma.

124. The method of any one of claims 47-121, wherein the hematologic cancer is a myeloproliferative neoplasm (MPN).

125. The method of claim 120, wherein the myeloproliferative neoplasm is chronic myelogenous leukemia (CML), polycythemia vera (PV), primary myelofibrosis, essential thrombocythemia, chronic neutrophilic leukemia, or chronic eosinophilic leukemia.

126. The method of any one of claims 1-125, wherein the method further comprises administering an additional MAPK pathway inhibitor.

127. The method of claim 126, wherein the additional MAPK pathway inhibitor is a KRAS inhibitor, NRAS inhibitor, HRAS inhibitor, PDGFRA inhibitor, PDGFRB inhibitor, MET inhibitor, FGFR inhibitor, ALK inhibitor, ROS1 inhibitor, TRKA inhibitor, TRKB inhibitor, TRKC inhibitor, EGFR inhibitor, IGFR1R inhibitor, GRB2 inhibitor, SOS inhibitor, ARAF inhibitor, BRAF inhibitor, RAF1 inhibitor, MEK1 inhibitor, MEK2 inhibitor, c-Mycv, CDK4/6, inhibitor CDK2 inhibitor, FLT3 inhibitor, or ERK1/2 inhibitor.

128. The method of claim 127, wherein the additional MAPK pathway inhibitor is a KRAS inhibitor.

129. The method of claim 127, wherein the additional MAPK pathway inhibitor is a BRAF inhibitor.

130. The method of claim 127, wherein the additional MAPK pathway inhibitor is an EGFR inhibitor.

131. The method of claim 127, wherein the additional MAPK pathway inhibitor is a CDK4/6.

132. The method of claim 127, wherein the additional MAPK pathway inhibitor is a FLT3 inhibitor.

133. The method of claim 127, wherein the additional MAPK pathway inhibitor is adagrasib, afatinib, ASTX029, binimetinib, cetuximab, cobimetinib, dabrafenib, dacomitinib, encorafenib, erlotinib, gefitinib, gilteritinib, lapatinib, LTT462, LY3214996, necitumumab, neratinib, nimotuzumab, osimertinib, panitumumab, selumetinib, sotorasib, trametinib, ulixertinib, vandetanib, or vemurafenib.

134. The method of claim 127, wherein the additional MAPK pathway inhibitor is adagrasib.

135. The method of claim 127, wherein the additional MAPK pathway inhibitor is cetuximab.

136. The method of claim 127, wherein the additional MAPK pathway inhibitor is dabrafenib.

137. The method of claim 127, wherein the additional MAPK pathway inhibitor is encorafenib.

138. The method of claim 127, wherein the additional MAPK pathway inhibitor is gilteritinib.

139. The method of claim 127, wherein the additional MAPK pathway inhibitor is palbociclib.

140. The method of claim 127, wherein the additional MAPK pathway inhibitor is panitumumab.

141. The method of claim 127, wherein the additional MAPK pathway inhibitor is sotorasib.
